(19) United States
(12) Patent Application Publication
(10) Pub. No.: US 2025/0205275 A1
(43) Pub. Date: Jun. 26, 2025

(54) POLYMERIC IRON BINDING COMPOSITIONS AND METHODS OF USE FOR TREATING DISEASES ASSOCIATED WITH EXCESS DAMAGING REACTIVE IRON

(71) Applicant: Fe Pharmaceuticals (Canada) Inc., Guelph (CA)

(72) Inventors: Bruce E. Holbein, Guelph (CA); M. Trisha C. Ang, Scarborough (CA); Roger Gumbau-Brisa, Sydney (CA)

(73) Assignee: Fe Pharmaceuticals (Canada) Inc., Guelph, ON (CA)

(21) Appl. No.: 18/849,702

(22) PCT Filed: Mar. 24, 2023
(86) PCT No.: PCT/CA2023/050396

§ 371 Date: Sep. 23, 2024

Related U.S. Application Data
(60) Provisional application No. 63/323,304, filed on Mar. 24, 2022.

(51) Int. Cl. A61K 31/79; A61K 31/4422; A61P 39/04

(52) U.S. Cl. CPC A61K 31/79; A61K 31/4422; A61P 39/04

(57) ABSTRACT

Disclosed herein are iron-chelating polymers for treatment of diseases with an iron-mediated pathology, said polymers comprising a reaction product of a first monomer unit and a second monomer units polymerized by a reversible addition-fragmentation chain transfer mechanism with the use of a suitable addition-fragmentation chain transfer agent, wherein: the first monomer unit is represented by Compound (I);

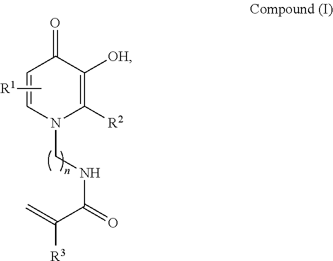

wherein the second monomer unit is independently selected from the group consisting of 1-vinyl-2-pyrrolidone, acrylic acid, methyl methacrylate, N,N-dimethyl-acrylamide, ethyl methacrylate, N-vinyl imidazole and styrene; the iron-chelating polymer is dissolved in an aqueous medium; the iron-chelating polymer has a molecular weight prior to chelation of at least about 1500 Da; and the iron-chelating polymer comprises one or more intramolecular hexadentate ligands for chelating iron. The described polymers are useful in the treatment of diseases associates with excess reactive iron.

### RELATED APPLICATIONS

The present application claims priority to U.S. Provisional Patent Application 63/323,304 filed Mar. 24, 2022 herein incorporated in its entirety.

### FIELD

The present disclosure relates to methods and uses of iron-chelating; more specifically, methods and uses for treatment of diseases associated with excess damaging reactive iron.

### BACKGROUND

### Iron as a Biological Catalyst and Iron Excess Toxicity

Iron is a trace essential nutrient metal used by all vertebrate animals as it provides an active catalytic center for a range of enzymes involved in DNA synthesis, metabolism and cell defense and, with few exceptions, it cannot be replaced by alternative trace metals. Around 2% of human genes encode for iron-proteins with more than half of these having catalytic function with oxidoreductase class enzymes being the largest fraction (Andreini et al., 2018).

Table 1 provides examples of iron-dependent enzymes needed by most cells for critical functions at a number of physiological levels. These enzymes are involved in cell growth and replication and generally, iron cannot be substituted while retaining enzymatic activity.

TABLE 1
Iron-dependent enzymes

| Enzyme | Function | Reference |
|---|---|---|
| Cytochrome | Energy | Williams, |
| oxidase EC | production | 1987 |
| 7.1.1.9 | | |
| Citrate | TCA cycle | Beinert et |
| aconitase EC | | al, 1996 |
| 4.2.1.3 | | |
| Ribonucleotide | DNA | Jordan and |
| reductase EC | synthesis | Reichard, |
| 2.7.7.56 | | 1998 |
| DNA | DNA | Zhang et al, |
| polymerase | replication | |
| EC 2.7.7.7 | and repair | 2014 |
| Oxoglutarate | Lipid | McDonough |
| oxygenase EC | biosynthesis | et al, 2010 |
| 1.13.12.19 | | |
| Superoxide | Superoxide | Parker and |
| dismutase EC | radical | Blake, 1988 |
| 1.15.1.1 | detoxification | |
| Catalase EC | Peroxide | Alfonso- |
| 1.11.1.21 | detoxification | Prieto et al, |
| | | 2009 |

However, iron atoms may be toxic due to chemical reactions catalyzed by free reactive iron atoms that can generate reactive oxygen radical species (ROS) in aerobic systems at physiological pH values. These ROS chemical oxidizing radicals can damage cells and tissues leading to cell, tissue damage and disease.

Free reactive iron atoms may generate ROS through the use of superoxide. Superoxide (O₂⁻) is produced constitutively by the electron transport chain in cells by slippage of an electron to O₂ with concomitant reduction of Fe³⁺ to Fe²⁺. The human body produces around 5 g O₂⁻/day (Hayyan et al., 2016) but superoxide dismutase (an iron dependent enzyme) normally neutralizes quantities in excess of normal amounts as needed for cell regulation and signaling (Ray et al., 2012). Superoxide is also produced by nicotinamide adenine phosphate oxidase in the phagocytic defensive cells of vertebrate animals and this can be used to kill phagocytosed invading microbes (Minakami et al., 2006). Superoxide can set up a cascade cycling of Fe reduction/oxidation coupled to peroxide and hydroxyl radical ·OH production as shown below with ·OH being highly toxic through damage to DNA and membrane lipids (Halliwell et al., 2021).

Fe³⁺+·O₂\hat{ }→Fe²⁺+O₂

Fe²⁺+H₂O₂→Fe³⁺+OH⁻+·OH

### Total Reaction Net:

·O₂⁻+H₂O₂→O₂+OH⁻+·OH

Therefore, regulation of iron supplies in the body is important to ensure sufficient quantities of needed amounts of iron, yet also prevent accumulation of excess amounts of iron above normal amounts. As explained in further detail herein, vertebrate animals generally achieve this iron homeostasis by controlling amounts of iron taken up from food in the gut, carrying iron on transferrin which is a chaperone carrier protein which substantially keeps iron from reacting while it is delivered to cells for use and also by holding sufficient iron reserves within ferritin intracellular stores, where stored iron is substantially both spatially and chemically shielded and non-reactive.

Furthermore, because iron is also needed by pathogens that invade and infect the body there is an active iron withholding defense that occurs early during infection with a reduction in the amounts of circulating iron on transferrin so as to restrict access of iron as needed by pathogens for their growth. For a review of these infection-related aspects see Holbein et al., 2021, the entire contents of which are incorporated herein by reference.

### Chemical Containment and Regulation of Normal Iron Homeostasis

Containment of reactive iron and its ROS products may be achieved through both compartmentalization and its chemical chaperoning. Phagocytes including macrophages and polymorphonuclear leukocytes can compartmentalize ROS production within intracellular phagosomes which spares other intracellular organelles and the extracellular milieu from direct ROS exposure (Fang, 2004). The bulk of total body iron stores represents around 55 mg/kg and are located intracellularly, incorporated within hemoglobin (erythrocytes), myoglobin (muscle cells) and ferritin (liver cells) (Yiannikourides et al., 2019). Iron incorporated into heme and ferritin may not be freely chemically available to participate in ROS production. Body iron stores are shuttled to sites of use in other cells by transferrin which binds iron so that it can be chaperoned in circulation and not available for ROS reactions. There is an additional small labile iron pool located both intracellularly and extracellularly which is readily ROS reactive (Kakhlon et al., 2002) and this pool is relevant in relation to iron dysregulation which may increase the amounts in this labile pool.

Some currently used therapeutic agents including aminoglycoside antibiotics can bind iron not fully satisfying its chaperoning requirements (Ezraty et al., 2016) and can induce ROS related tissue damage (Li et al., 2009), likely due to the mobilization of labile iron. This underscores the need for containment and chaperoning of labile ROS reactive iron in the host as part of normal homeostatic control.

Of the 3750 mg of total iron in the human body, around 65% of this is incorporated into heme in erythrocytes, 10% within heme of myoglobin of the muscles, 14% in macrophage cells of the reticulo-endothelial system (RES), 28% stored within ferritin in hepatocytes and 4% in bone marrow cells (Yiannikourides et al., 2019). Iron taken up by enterocytes in the gut can be stored transiently in small amounts in ferritin until there is demand for iron replenishment elsewhere in the body and then transferrin can take up this iron into the bloodstream serving as a shuttle/delivery protein chaperone. The transferrin pool is normally only 30% saturated with iron and therefore it can provide iron holding capacity to limit amounts of labile non-transferrin-bound iron in circulation. The transferrin iron pool represents a flux of around 25 mg iron per day (Pantopoulos, 2018). The body has no excretory mechanism for iron and small daily losses of 1-2 mg from skin desquamation and other sources are correspondingly compensated by uptake from the gut.

Hepcidin, a 25-amino acid peptide hormone produced primarily by liver hepatocytes, is a regulator of iron homeostasis and it can negatively regulate iron uptake by inactivating plasma membrane bound ferroportin on enterocytes and macrophages cell membranes as needed for transfer of intracellular iron to extracellular compartments (Nemeth et al., 2022). This provides tight regulation of iron homeostasis which is involved in ensuring no excess toxic quantities of iron either intracellularly or extracellularly. Iron dysregulation often presents with elevated amounts of circulating plasma iron including transferrin bound iron with >50% saturation along with elevated non-transferrin-bound iron (NTBI) that can reach >0.5 μM (Vinchi, 2021). The labile portion of NTBI can play a role in the pathogenesis of the various non-microbial diseases discussed below while elevated plasma iron in the form of transferrin bound iron can also support microbial infection (Holbein et al., 2021).

### Iron Dysregulation Underlying Disease

### The Nature of Iron Dysregulation

Iron dysregulation diseases can result from increased amounts of circulating labile reactive plasma iron. This can be evidenced by elevated plasma transferrin bound iron (TBI) with transferrin saturation above its normal 30%, sometimes reaching 100% saturation (Akinc et al., 2011). With high saturation of transferrin, some of the plasma iron may not be effectively chaperoned and this can provide a pool of non-transferrin-bound iron (NTBI) labile reactive iron (LPI). This LPI is also more mobile and capable of entering cells where it is toxic (Cabantchik et al., 2005; Cabantchik, 2014).

While the iron dependence for supporting growth of microbial pathogens or cancer cells due to their increased needs for iron is now well established, there is increasing evidence that dysregulation of normal iron homeostasis in vertebrate animals resulting in above normal amounts of free iron that is unprotected/non-chaperoned and therefore chemically reactive, in some iron pools and compartments of the body, is involved in the pathogenesis of other diseases. Diseases involving iron dysregulation may not be directly related to iron needs for growth of pathogenic microbial or cancer cells.

Excess plasma iron in the form of TBI and NTBI LPI can influence two main categories of diseases. Elevated plasma iron e.g. in the form of transferrin-iron can support cell proliferative diseases including infection and cancer. Increased plasma iron present as non-transferrin bound LPI can be part of the pathology of other diseases where excess reactive iron triggers iron-related pathologies.

Examples of various diseases from both categories are summarized in Tables 2 and 3 below:

TABLE 2
Diseases with iron-dysregulation

| Disease Category | Disease | Example | Reference |
|---|---|---|---|
| Proliferative Cell | Microbial | Staphylococcal, | Holbein et al, |
| Replication | Infection | Candidiasis | 2021 |
| | Bacterial Sepsis | Pseudomonas | Bullen et al, |
| | | aeruginosa, | |
| | | Neisseria meningitidis | 2000 |
| | Parasitic Infection | Chagas disease, malaria | Mach and Sutak, |
| | | | 2020 |
| | Viral | HIV, HBV, HCV, | Schmidt, 2020 |
| | Infection | HCMV | |
| | Cancer | Pancreatic, Liver, Lung | Torti et al, 2018 |
| Iron-mediated Pathology | Diabetes | Type 2 Diabetes | Harrison et al, |
| | | | 2023 |
| | Cardiovascular | Cardiomyopathy | Li and Zhang |
| | | | 2021 |
| | Neurological | Alzheimer's, ALS, | David et al, 2022 |
| | | MS, Parkinson's | |
| | Lung Fibrosis | Idiopathic Pulmonary | Ogger and |
| | | Fibrosis | Byme, 2020 |
| | Autoimmune | Rheumatoid Arthritis, | Baker and Ghio, |
| | | Lupus Erythematosus | 2009 |
| | Kidney | Fanconi Syndrome | Smith and |
| | | | Thévenod, 2009 |
| | Retinal | Age-Related Macular | Loh et al, 2009 |
| | | degeneration | |
| | Iron Overload | Hemochromatosis | Pantopoulos, |
| | | | 2018 |
| | Cirrhosis | Non-alcoholic fatty | Chen, 2022 |
| | | cirrhosis | |

TABLE 3
Examples of diseases with at least an aspect linked to iron dysregulation
with ROS mediated cell and tissue damage as caused by excess amounts
of labile reactive iron (i.e. diseases with an iron-mediated pathology),
with a supporting reference as to ROS damage

| | | Excess Fe |
|---|---|---|
| | | ROS |
| | Main Class | implicated |
| Disease Name | of Disease | reference |
| Systemic Lupus | Autoimmune | Theut LR et |
| including | | al, 2020 |
| associated | | |
| nephritis | | |
| Rheumatoid | Autoimmune | Pretorius E |
| Arthritis | | et al, 2017 |
| Parkinson's | Neurological | Schumacher- |
| Disease | | Schuh MS et |
| | | al., 2016 |
| Alzheimer's | Neurological | Liu J-L et al, |
| Disease | | 2018 |
| Fredreich's | Neurological | Hadzhieva |
| Ataxia | | M, et al, |
| | | 2014 |
| Amyotrophic | Neurological | Hadzhieva |
| lateral sclerosis | | M, et al, |
| | | 2014 |
| Fanconi's and | Genetic, | Smith CP |
| related kidney | metabolic | and F |
| disease | | Thévenod, |
| | | 2009 |
| Type 2 Diabetes | Genetic, | Padmanaban |
| Mellitus | metabolic | Venkatesan |
| | | et al, 2021 |
| Hemochromatosis | Genetic- | Joshi et al, |
| | excess iron | 2021 |
| | uptake | |
| Thalassemia | Genetic - | Fibach and |
| | excess | Dana, 2019 |
| | uptake and | |
| | transfusional | |
| | iron overload | |
| Cystic Fibrosis | Genetic | Maniam P., |
| | | et al, 2021 |
| Macular | Inflammatory | Ueda K., et |
| Degeneration Eye | | al, 2018 |
| Disease | | |
| Cardiovascular | Inflammatory | Li S and |
| Disease | | Zhang X, |
| | | 2021 |

A brief review of various diseases associated with iron-dysregulation is provided below.

Infections—All pathogenic microorganisms with the exception of Borrelia burgdorferi, the agent of Lyme disease, have requirements for iron for their growth and replication in the human body (Schaible et al., 2004). B. burgdorferi can utilize manganese in place of iron (Troxell et al., 2013). The vertebrate host normally maintains conditions of low iron bioavailability to microbes including in extracellular compartments such as plasma, respiratory secretions and tears where infection can be initiated, thus providing a natural nutritional immunity. Transferrin in plasma and lactoferrin in tears and other secretions can maintain very low levels of freely available iron (Murdoch et al., 2022). Lactoferrin concentrations can reach >3 mg/mL in tears (Hanstock et al., 2019). Furthermore, when infection is first detected the body can mount an early iron withdrawal defense response where extracellular iron concentrations are further reduced by moving this iron to intracellular stores (Nemeth et al., 2022). Successful pathogens can deploy a variety of virulence mechanisms to compete for iron as reviewed elsewhere (Holbein et al., 2021).

Pathogenic microbes—Iron has been found to support infection by a number of pathogenic microbes and Table 4 summarizes some microbial infections in relation to the main host iron sources feeding the infection. Of these, some have cell wall surface receptors that can intercept host iron sources while others can deploy high affinity siderophores to strip iron from host transferrin or lactoferrin (Holbein et al., 2021).

TABLE 4
Iron sources for pathogenic microbes

| Microbial Pathogen | Host iron source | Reference |
|---|---|---|
| Neisseria meningitidis | Transferrin | Holbein, 1981 |
| Pseudomonas | Transferrin, | Xiao and Kisaalita, 1997 |
| aeruginosa | Lactoferrin | |
| Acinetobacter | Transferrin, | Yamamoto et al, 1999; |
| baumannii | Lactoferrin, heme | Giardina et al, 2019 |
| Campylobacter jejuni | Transferrin, | Miller et al, 2008 |
| | Lactoferrin | |
| Staphylococcus aureus | Transferrin, heme | Modun et al, 1998; |
| | | Skaar and |
| | | Schneewind, 2004 |
| Mycobacterium | Transferrin | Clemens et al, 1996 |
| tuberculosis | | |
| Candida albicans | Transferrin | Knight et al, 2005 |
| Aspergillus fumigatus | Transferrin | Hissen et al, 2004 |

Bacterial sepsis—Sepsis is a life threatening condition killing around 11 million people worldwide annually (Olwal et al., 2021). It can result from a severely dysregulated host response to inflammation. Sepsis can develop form viral infections such as COVID-19 (Olwal et al., 2021) as well as through microbial infection. Bacterial sepsis can develop from serious bacterial infection with the host inflammatory response over stimulated and becoming dysregulated (Liu et al., 2021), this often triggered by bacterial cellular components released during infection including endotoxin (Lehmann et al., 2015).

During sepsis, iron metabolism can be altered with increased iron uptake into cells and this has been associated with increased iron-driven oxidative injury and cell death (Liu et al., 2021). High serum iron levels are also associated with sepsis and poor sepsis outcomes (Lan et al., 2018). Transferrin saturation reflecting serum iron availability is correlated to sepsis outcomes with increased iron availability pronounced in lethally ill subjects (Liu et al., 2021; Tacke et al., 2016). This aspect as driven by excess iron is additional to iron feeding microbial infection as it relates to host dysregulated inflammation. Thus, the condition of iron dysregulation can provide for conditions of increased iron availability to support rapidly proliferating microbes on the one hand and increased host damage from the associated dysregulated inflammatory response on the other hand.

Parasitic pathogens—Multicellular parasitic organisms may also require iron to invade and mount infection in vertebrate hosts (Mach et al., 2020). Leishmania chagasi which causes leishmaiasis can utilize iron from transferrin, lactoferrin or heme when growing in its promastigote form, taking these up directly without deployment of iron intercepting siderophores (Wilson et al., 1994). Trypanosoma brucei can employ transferrin surface receptors and Entamoeba histolytica can employ lactoferrin receptors for iron acquisition (Mach et al., 2020). Plasmodium falciparum which causes malaria has also been shown to possess surface receptors for transferrin-iron which resides within hemoglobin-rich erythrocytes (Clark et al., 2014). Addition of iron has been shown to promote infection with Trypanosoma cruzi (Lalonde et al., 1984) and iron supplementation has been associated with increased risk to malaria (Clark et al., 2014). Parasitic infection in contrast to most microbial infections can be chronic in nature and has been associated with what is described as the anemia of chronic disease (Wicinski et al., 2020). This can also occur in cancer and this chronic anemia may be considered the result of the prolonged effort of the host to restrict iron to the invading pathogens or cancer cells. Host iron deficiency has been found to be protective against malaria, based on epidemiological studies (Clark et al., 2014).

Viral infections—Viral replication in host cells can be affected by host iron in the case of both DNA viruses such as hepatitis B (HBV) and human cytomegalovirus (HCMV) and RNA viruses, such as hepatitis C (HCV) and human immunodeficiency virus (HIV) (Schmidt, 2020). It remains unclear as to whether iron dysregulation predisposes the host to these viral infections or alternatively, if the viral infection leads to host iron dysregulation. Elevated iron levels are associated with progression of chronic HBV infection (Wei et al., 2018) and iron addition has been shown to enhance HCV viral replication in vitro (Kakizaki et al., 2000). Transferrin receptor-1 mRNA levels were increased due to HIV infection, leading to an increased iron uptake and higher level of cellular iron (Chang et al., 2015). However, it has been suggested that in cirrhotic subjects, HBV related liver injury, but not the HBV infection itself may be cause changes in serum iron markers (Mao et al., 2015).

Cancer—The role of iron and its dysregulation in cancer is multi-faceted as reviewed by Torti et al., (2018). Stevens et al., (1994) reported on a cohort of 14,000 US National Health and Nutrition survey participants and found that participants with higher transferrin (Tf) iron saturation levels were at higher risk of cancer than participants with lower transferrin Tf saturation levels, a finding supported by subsequent studies (Wu et al., 2004). Cancer cells arising from various tissues such as breast or liver can have increased need for iron compared to normal cells and produce increase amounts of cell surface transferrin receptors in response to their increased and continuous iron needs (Greenshields et al., 2019). Targeting transferrin receptors has been proposed for its therapeutic potential (Shen et al., 2018) as shown using antibodies directed to cancer cell surface transferrin receptor TfR1 (Candelaria et al., 2021). This aspect has appeal as common features to all cancers are possession of TfR1 receptors (Candelaria et al., 2021), their altered iron metabolism and increased needs for iron to support their rapid growth (Zhang et al., 2020) and their primary dependence on host transferrin as the source of this iron. Thus, any advances in regard to beneficial iron restriction could apply to various cancers broadly.

Cancer can also induce a chronic anemia as seen in other chronic diseases and this appears to be a host attempt to restrict cancer growth (Greenshields et al., 2019). Somewhat paradoxically, cancer cells are more susceptible to ferroptosis, a programmed cell death triggered by excess reactive iron (Lei et al., 2019) suggesting host iron withdrawal defense to restrict cancer growth could also impede cancer cell killing via ferroptosis.

Ferroptotic cell death—Ferroptosis is an additional form of regulated cell death (RCD) but unlike other forms such as apoptosis and necrosis it can be caused by iron-dependent accumulation of lipid peroxides which kill cells, although it shares other common features with the other modes of RCD (Lei et al., 2019). ROS produced through the iron-catalyzed Fenton reaction can contribute to its initiation (Toyokuni et al., 2020) and therefore iron dysregulation with its excess labile iron can be a predisposing factor.

Potential inhibitors of ferroptosis have been investigated including ROS-trapping antioxidants such as α-tocopherol and ferrostatin-1 and these have shown potential for reducing ferroptotic damage (Angeli et al., 2017).

Excess labile iron driving ferroptosis has also been demonstrated through its suppression by addition of the iron chelator deferoxamine (Yang et al., 2008). However, ferric-nitriloacetate (NTA) has been shown to promote ferroptosis and renal carcinoma by driving Fenton activity (Toyokuni et al., 2020). Various iron chelators can differ in their abilities to fully coordinate and therefore fully chaperone iron (Holbein et al., 2021). Deferoxamine can fully hexadentate-coordinate iron within a single deferoxamine molecule while NTA requires two chelator molecules to fully satisfy a single iron atom. Therefore, depending on the prevailing chelator and iron concentrations, incompletely coordinated, Fenton-reactive species can be formed which appears to underlie the toxicity of some of the currently used medical chelators (Holbein et al., 2021). Further, small molecule cell-permeable chelators can more readily penetrate cells reaching cellular iron stores and mobilize additional labile iron supplies, which can in turn exacerbate iron dysregulation.

The excess supply of labile reactive iron observed as a feature of iron dysregulation may underpin tissue toxicity with a number of diseases and ferroptosis appears to be at least part of the pathology of these diseases, as further discussed below.

Inflammatory diseases—While inflammation typically accompanies infection and cancer, other diseases appear to be primarily inflammatory in nature and iron dysregulation and its associated ROS activity can be involved in the inflammatory response.

Ocular—Iron dysregulation of the eye has been linked to various eye diseases affecting the cornea and retina including corneal epithelial disease, corneal endothelial cell dysfunction, retinal pigment epithelial (RPE)-associated eye diseases, glaucoma, diabetic retinopathy (DR), retinal ischemia/reperfusion injury (RIRI), retinoblastoma, retinitis pigmentosa (RP), and age-related cataracts and common to these, ferroptosis pathology has been implicated (Zhang et al., 2022). Ferroptotic iron induced toxicity through ROS damage can be linked to corneal diseases such as cataractogenesis, inflammatory retinal diseases such as age related macular degeneration (AMD) and optic neuropathies (Loh et al., 2009). Transferrin mRNA levels can be upregulated in AMD possibly in response to increased retinal iron loads (Chowers et al., 2006). This feature can provide a potential avenue to new therapeutics including the use iron chelators to treat eye diseases that are in urgent need of new therapeutics.

Pulmonary iron content is regulated as excess iron can catalyze ROS formation and this has been linked to pathogenesis of chronic inflammatory lung diseases such as idiopathic pulmonary fibrosis (Ogger et al., 2020). Iron accumulation was increased in lung sections from subjects with IPF and human lung fibroblasts show greater proliferation and cytokine and extracellular matrix responses when exposed to increased iron levels (Ali et al., 2020). These authors provided direct evidence for iron overload affecting the progression of pulmonary fibrosis. They investigated whether changes in iron homeostasis are the cause or a consequence of pulmonary fibrosis and used mouse models of iron overload to show that iron accumulation results in impaired lung function and subsequently worse pulmonary fibrosis upon lung injury by bleomycin (Ali et al., 2020). Lung fibrosis is a progressive irreversible disease as fibrotic lung tissue does not repair/remodel making anti-fibrotic agents an urgent need. Overcoming iron dysregulation to reduce iron driven fibrosis is a new potential avenue for therapy. Iron chelation therapeutics have potential for use in slowing or stopping pulmonary fibrosis.

Kidney—Increased kidney proximal tubule (PT) cell cytosolic non-transferrin Tf bound, i.e., labile iron has been shown to induce generation of ROS in PT cells and this could contribute to the progression of proteinuric chronic kidney diseases (Smith et al., 2009). Recent clinical studies using anti-oxidative drugs that can reduce labile iron suggest that chelation of iron in the kidney has beneficial effects on the course of chronic kidney disease (Swaminathan et al., 2008). Nephritic syndromes associated with damage to the glomerular filter, urinary Tf concentration can increase and may even lead to hypotransferrinemia, iron loss and microcytic anemia (Prinsen et al., 2001).

Nephritic kidney damage with urinary transferrin excretion is also part of the pathology associated with autoimmune diseases including lupus erythematosus (Theut et al., 2020) and rheumatoid arthritis (Kochi et al., 2018) as further discussed further below.

Diabetes—High dietary iron and dysregulated iron metabolism can be risk factors for type 2 diabetes mellitus (T2DM) and can affect most of its features: of decreased insulin secretion, insulin resistance, and increased hepatic gluconeogenesis (Harrison et al., 2023). Dysregulated iron metabolism with increased serum levels of ferritin have been found in newly diagnosed type 2 diabetes but not in individuals with pre-diabetes (Venkatesan et al., 2021). Oxidative stress from ROS is now known to be an underlying mediator of diabetic complications (Giacco et al., 2010). Iron and ferroptosis have been shown to participate in pancreatic beta cell death (Li J. et al., 2020). In model studies using T2DM, mice insulin secretion was worsened by ferroptosis-inducing compounds. However, quercetin (a natural iron chelator), ferroptosis inhibitor ferrostatin-1 and iron-chelating deferoxamine, each rescued cell viability when cells were challenged with high-glucose (Li D. et al., 2020). These studies support the potential for use of iron chelating therapeutics in treatment of T2DM.

Cardiovascular—Ferroptosis driven by iron dysregulation has been implicated in several cardiovascular disease conditions including cardiomyopathy, atherosclerotic disease and myocardial ischemia/perfusion injury (Li et al., 2021; Fang et al., 2023). Inhibition of ferroptosis by ferrostatin-1 improved cardiac function and reduced mortality in a doxorubicin-induced mouse model of cardiomyopathy, which was associated with the release of free cellular iron caused by HO-1 upregulation (Fang et al., 2019). However, the mechanisms of ferroptosis in heart and vasculature disease remain elusive (Li et al., 2021).

Autoimmune—The role of iron regulation in immune-related diseases was recently reviewed by Cronin et al., (2019). Substantial evidence has now linked iron dysregulation to the pathogenesis of lupus erythematosus (Wincup et al., 2021). Lupus nephritis has been linked to renal iron accumulation (Theut et al., 2020) and ferroptosis kidney cell damage has been described as an important feature of its pathology (Wincup et al., 2021).

Neurological—Many neurological conditions have been shown to coincide with altered bodily distribution of various transition series biometals, especially in the case of iron (Pfaender et al., 2014). Iron dysregulation with increased labile plasma iron supply and resulting increased ROS, creating oxidative stress and damage on neurological tissues has now been linked to Freidreich's ataxia, Alzheimer's disease (AD), Parkinson's disease, Multiple Sclerosis (MS) and Amyotrophic Lateral Sclerosis (ALS) (David et al., 2022). Increased ferroptosis with corresponding destruction of neurological cells has been described in Parkinson's disease and MS (Hadzhieva et al., 2014) as well as for Alzheimer's disease (Das et al., 2021).

For example, Parkinson's disease (PD) can be characterized by progressive motor impairment attributed to progressive loss of dopaminergic neurons in the substantia nigra (SN) pars compacta. In addition to an accumulation of iron, there can also be an increased production of reactive oxygen/nitrogen species (ROS/RNS) and inflammatory markers seen in this pathology (Medeiros et al., 2016). In addition, abnormal iron increases can be detected in AD subjects, although controversy continues regarding iron's association with AD plaques (Das et al., 2021). It appears that a common feature of iron dysregulation underlying neurological diseases is iron driven ferroptosis neurological cell death (Ren et al., 2020).

Iron overload—While body iron overload can follow repeated blood transfusions as seen in subjects with Thalassemia, i.e., because the body lacks an excretory pathway for excess iron, congenital disorders of iron overload such as hemochromatosis can represent iron overload disorders caused by dysregulated iron homeostasis. In hemochromatosis, disruption of the hepcidin pathway due to mutations in genes encoding auxiliary factors in iron signaling to hepcidin, can result in insufficient hepcidin responses to iron intake or to high body iron stores. This can cause loss of hepcidin mediated feedback inhibition in dietary iron absorption and consequently unregulated uptake of dietary iron, elevated transferrin iron saturation and appearance of labile reactive non transferrin bound iron (Pantopoulos, 2018).

Cirrhosis—Non-alcoholic fatty liver disease (NAFLD) is a chronic liver disease, beginning with the presence of >5% excessive lipid accumulation in the liver, and typically developing into non-alcoholic steatohepatitis, fibrosis, cirrhosis and often hepatocellular carcinoma (Chen, 2022). Excess free reactive iron and its associated ROS-mediated tissue damage have been associated with severity of NAFLD (Chen, 2022).

Anemia of chronic infection and inflammation—The anemia of inflammation (AI) or often referred to as the anemia of chronic disease (ACD) is a secondary anemia that can develop slowly as a result of chronic inflammation during chronic infection (especially parasitic), cancer and with at least some of the other inflammatory diseases, such as diabetes and autoimmune diseases (Wicinski et al., 2020; Ismaiel et al., 2020; Weiss et al., 2019). Overall, it appears to be a host defensive mechanism. A short term hypoferremia response can often be seen early in the acute stages of infection and this has also been shown to be triggered by inflammatory mediators such as ILK-6 and demonstrated to be a relatively short term active mechanism for restricting iron supply to growing invaders (Holbein et al., 2021). Identification of subjects with concomitant true iron deficiency can present a diagnostic challenge in AI/ACD because they may need specific evaluation for the source of blood loss and iron-targeted management strategies (Weiss et al., 2019).

### Therapeutic Options

### Iron Restriction

Higher heme iron intake and increased body iron stores were associated with a greater risk of Type 2 diabetes as shown in a meta-analysis of 11 prospective studies (Bao et al., 2012). Blood-letting to reduce overall body iron stores has shown potential benefit in the treatment of diabetes (Fernández-Real et al., 2002).

In principle, restriction of iron uptake can lower amounts of deleterious liable reactive iron. However, withholding (or supplying) iron has remained controversial in medicine over the years due to the delicate balance required for iron homeostasis and the consequences of triggering anemia. While supply of dietary iron would be safer, administration of parenteral iron to treat anemia, especially the anemia of chronic disease has serious implications. Given the roles of dysregulated iron in infection and other diseases this requires careful consideration and likely should be avoided if possible.

### Iron Chelators

Compositions to chelate and withhold iron in the extracellular environment of growing cells so as to restrict their uptake of iron as needed for use in growth within the cells and therefore affect cell growth have been previously disclosed. These disclosures may be found for example in U.S. Pat. No. 10,709,784, entitled “Metal chelating compositions and methods for controlling the growth or activities of a living cell or organism” and in U.S. Pat. No. 11,059,785, entitled “Polymeric metal chelating compositions and methods of preparing same for controlling growth and activities of living cells”, both of which are incorporated herein in their entireties by reference. Such compositions have been shown in various examples as reviewed by Holbein et al, 2021, to assist the natural iron withdrawal defenses of vertebrate animals for fighting diseases where iron supply in the body promotes growth a pathogen, i.e., of either a microbial invading pathogen or a cancer cell with non-controlled growth arising within the body of an animal.

These various disclosures referenced above have shown that application of an iron chelating composition that is soluble in aqueous media and with a molecular weight sufficiently large (i.e., nominally about >1500 Da) so as not to be normally taken up internally by living cells but that can bind iron in the environment of a living pathogenic cell, can withhold supply of essential iron as needed for growth in the intracellular aspects of the pathogenic cell. Thus, these compositions have been shown to address diseases in animals where iron supplies play a vital role in the growth, such as proliferation and pathogenesis of invading pathogen microbes or rapidly proliferating pathogen cancer cells arising from within the body.

These disclosures have also shown that low molecular weight iron chelators of a molecular weight of about <1500 Da such as those now used clinically to treat iron overload diseases in humans (such as Thalassemia) and that can more readily permeate into animal cells are not sufficiently effective for restricting growth of pathogenic microbial or cancer cells and also often display unacceptable toxicities to the animal being treated. Table 5 adapted from Holbein et al 2021 summarizes properties and compares and contrasts the current clinically approved chelators for use in humans as well as the proposed clinical chelators SP-420 and in comparison to DIBI. DIBI is an example of a composition prepared as disclosed in U.S. Pat. Nos. 10,709,784 and 11,059,785. The chemical structures of these chelators are shown below.

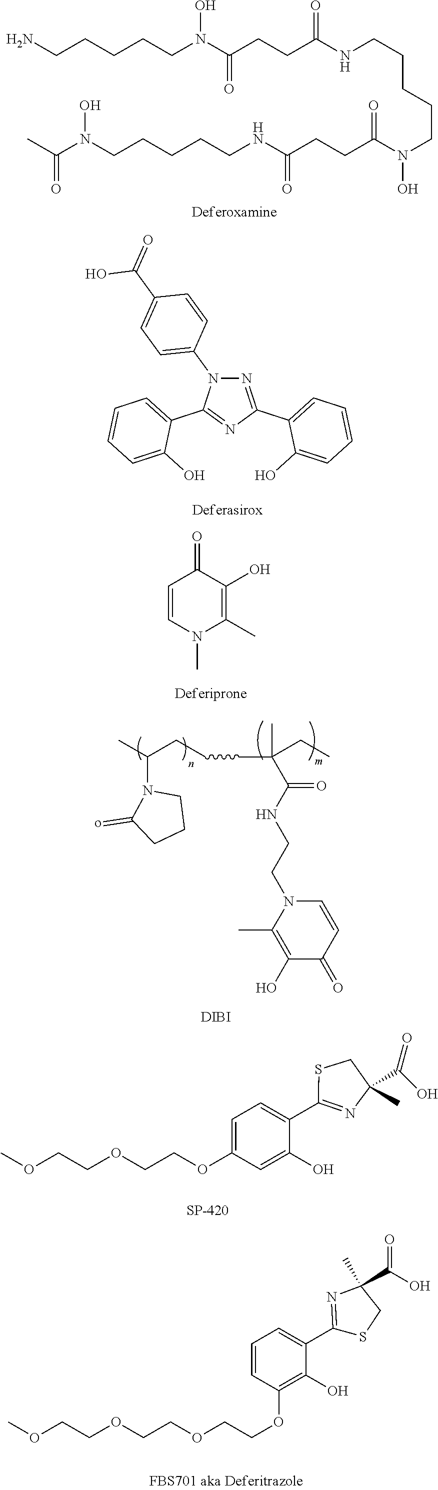

TABLE 5
Characteristics of conventional clinically used chelators; comparison to SP-420 and

| | Deferoxamine | Deferasirox | | Deferiprone | |
|---|---|---|---|---|---|
| Iron Chelator | (DFO) | (DEF) | SP-420 | (DFP) | DIBI |
| MW (Da) | 561 | 373 | 355.41 | 139 | 9,000 avg. ^{a} |
| | | | | | (polymer) |
| Fe(III): | 1:1 | 1:2 | 1:2 | 1:3 | 3:1 ^{a} |
| chelator | | | | | |
| complex | | | | | |
| Fe binding | 30.6 ^{b} | 36.5 ^{b} | Approx | 36.7 ^{a} | 41.0 ^{a} |
| constant | | | 33.1-33.4 | | |
| log K | | | based on | | |
| | | | close | | |
| | | | analogue | | |
| | | | 36 ^{q} and | | |
| | | | FBS0701^{r} | | |
| Hydrophobicity/ | −3 ^{b} | 4.3 ^{b} | −0.89 | −0.8 ^{b} | −1.87 ^{c} |
| hydrophilicity | | | | | |
| log P | | | | | |
| FDA Approval | 1968 | 2005 | in | 2011 | in |
| | | | development | | development |
| Treatment | transfusional | transfusional | transfusional | transfusional | anti- |
| Indication | iron | iron | iron | iron overload ^{d} | infective^{e} |
| | overload ^{d} | overload ^{d} | overload | | anti- |
| | | | | | inflammatory ^{f} |
| Serious | promotes | renal | renal | agranulocytos | None to |
| limitations and | infections ^{g} | failure/ | toxicity | is ^{h} | date from |
| toxicities | | Fanconi | Fanconi | | oral and |
| | | syndrome ^{h, i} | syndrome ^{s} | | systemic |
| | | | | | animal |
| | | | | | testing ^{j} |
| Antimicrobial | Poor, | Low | ND but | Very Low | Strong |
| activity: | promotes | | expected | (host toxic | |
| | growth as a | | low as | range) | |
| | microbial | | based on a | | |
| | siderophore | | microbial | | |
| | | | siderophore | | |
| MIC (μM) | >913^{k} | 21^{l} | ND | 575^{e} | 0.2^{e} |
| Gram positive | | | | | |
| bacteria | | | | | |
| MIC (μM) | >913^{k} | 133^{m} | ND | 287^{k} | 0.2^{o} |
| Gram negative | | | | | |
| bacteria | | | | | |
| MIC (μM) | >228ⁿ | 67^{p} | ND | 1151ⁿ | 0.2ⁿ |
| fungi | | | | | |

^{a} Gumbau-Brisa et al., 2020;
^{b} Vlachodimitropoulou et al., 2017;
^{c} Holbein, 2018;
^{d} Foley & Simeonov, 2012;
^{e}Parquet et al., 2018;
^{f} Fokam, et al., 2020;
^{g} Holbein et al., 2021;
^{h} Zeidan & Griffiths, 2018;
ⁱ Badeli et al., 2019;
^{j}Holbein, 2018;
^{k}Thompson et al., 2012;
^{l}Neupane & Kim, 2010;
^{m}Luo et al., 2014;
ⁿSavage et al., 2018;
^{o}Parquet et al., 2019;
^{p}Ibrahim et al., 2010;
^{q} Bergeron, Raymond J., et al. 2014;
^{r}Hider, Robert C., et al. 2015;
^{s} Taher, Ali T., et al. 2017.

Liu et al, 2010 (U.S. Pat. No. 8,334,320) disclosed polystyrene nanoparticles with an iron chelator moiety such as deferiprone or deferoxamine covalently linked to the surface of the nano particle for use in treating osteoblasts so as to limit oxidative damage and for the potential treatment of Parkinson's, Alzheimer's and Friedreich's Ataxia disease. However, the disclosed nanoparticles are insoluble and successful treatment from such insoluble compositions has not been reported. The macromolecular insoluble compositions may be ineffective for administration into a human or other animal and they may not be able to reach the immediate environment surrounding disease affected cells, i.e., so as to address excess iron in the immediate environment or within disease affected cells.

Attempts to use small molecule iron chelators such as the ones reviewed by Holbein et al 2021 and as described above, including deferiprone for the treatment of for example, experimental retinal disease (Ueda, K., et al, 2018), or newer experimental ones such as FBS0701 for treatment of for example, diabetes (Cooksey et al. 2010) have generally not provided satisfactory results in part related to the reported toxicities of the small molecules chelators used.

A newer small molecule chelator SP-420 structurally related to FBS701 (see above) and developed to overcome renal toxicity and other limitations of FBS701 and other similar molecules (Bergeron, et al, 2014) has still shown undesirable renal toxicity in clinical trials (Taher, et al, 2017). Such toxicity issues can seriously limit dosage, routes of administration to the body and frequency of administration such that these limitations seriously compromise treatment efficacy.

Additionally, iron chelators that are themselves, or are derived from microbial siderophores, i.e. as to their component iron binding sites, such as SP-420 which is derived from the siderophore desferrithiocin, may undesirably promote microbial infection if used as iron chelators to treat iron related diseases in humans. For example, this serious limitation was uncovered through the use of deferoxamine that is also a microbial siderophore as a chelator, to relieve iron overload in humans (see Table 5).

A number of clinical trials of natural products with iron chelating properties such as curcumin and polyphenolics have shown promise for lowering excess iron (Xu et al., 2021) but none have been approved for clinical use.

Hepcidin mimetics, stimulators of its production and ferroportin inhibitors have seen early clinical stage testing as to their safety and potential efficacy (Casu et al., 2018). The ferroportin inhibitor VIT-2763 has advanced to phase II trials and it has shown both low toxicity and good potential for lowering serum iron levels (Taher et al., 2022). Other approaches of using hepcidin mimetics including PTG-300, rusfertide have shown promising phase III results. As of yet, none of these have received regulatory approval for ongoing clinical use (Verstovsek et al., 2021).

While deferoxamine is the only currently clinically used small molecule chelator that can fully coordinate iron on a single chelator molecule, its ability to support microbial growth and infection otherwise severely limits its use as summarized in Table 5. Attempts to use deferoxamine in vivo have resulted in fatal infections in test animals (Kemp et al, 1995).

The stability of iron bound by deferoxamine with respect to the reactivity of its bound iron in relation to generation ROS and causing cell and tissue damage has also been brought into question by the finding that a deferoxamine starch conjugate composition has been shown to induce oxidative damage to red blood cells (Niihara et al, 2000).

There exists a need to address one or more of the deficiencies in the art as outlined above.

### SUMMARY

In one embodiment, the present disclosure provides for a chelating composition soluble in an aqueous medium for chelating iron, said chelating composition comprising:
a carrier material; and
one or more suitable type or types of iron binding chemical groups affixed to or incorporated into the structure of the carrier material;
wherein the one or more suitable type or types of iron binding chemical groups are one or more of carboxyl, hydroxyl, phenolate, catecholate, hydroxamate, hydroxypyridinone and hydroxyphenyltriazole carboxyl types;
wherein the carrier material comprises vinylpyrrolidone, imidazole acrylamide or styrene;
wherein the chelating composition has a minimum molecular weight sufficiently large, nominally about >1500 Da molecular weight, so as not to be normally taken up into the intra-cellular aspects internal to the cell membrane of a living animal cell and is able to bind iron with up to full chemical coordination of the bound iron on or within a single molecule of the composition;
wherein the chelating composition binds iron and remains substantially soluble with its bound iron substantially in the external cellular environment of the cell thereby reducing uptake of iron into the intra-cellular aspects internal to the cell membrane of the living animal cell; and as a result, the external and the internal aspects of the cell membrane and the internal aspects of the cell underlying the cell membrane of the living animal cell are protected from chemically mediated damage as caused in whole or in part by excess, above normal, amounts of iron in either the external environment or internal to the cell membrane of the animal cell.

In a further embodiment of the chelating composition above, the composition is prepared from:
at least a first and a second monomer unit, wherein the first monomer unit comprises suitable metal binding chemical groups incorporated or affixed thereto optionally independently selected from the group consisting of carboxyl, hydroxyl, phenolate, catecholate, hydroxamate, hydroxypyridinone and hydroxyphenyltriazole;
wherein:
the first and second monomer units are polymerized by a reversible addition-fragmentation chain transfer mechanism with the use of a suitable addition-fragmentation chain transfer agent;
optionally, the first monomer unit is represented by Compound (I)

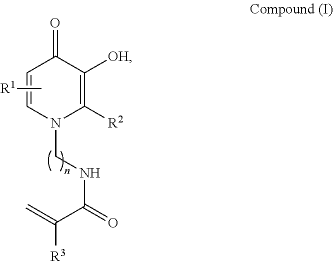

wherein
R¹ is independently selected from the group consisting of H, alkyl and alkyl optionally substituted with one of more of O, N or S;
R² is independently selected from the group consisting of H, alkyl and alkyl optionally substituted with one of more of O, N or S;
R³ is independently selected from the group consisting of H, alkyl and alkyl optionally substituted with one of more of O, N or S; and
n is 1 to 12;
optionally the second monomer unit is independently selected from the group consisting of 1-vinyl-2-pyrrolidone, acrylic acid, methyl methacrylate, N,N-dimethyl-acrylamide, ethyl methacrylate, N-vinyl imidazole and styrene.

In a further embodiment of the chelating composition above, the one or more suitable metal binding chemical groups are a hydroxypyridinone:

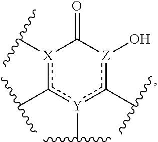

wherein X, Y and Z are independently N or C such that:
when X is N, Y and Z are C,
when Y is N, X and Z are C, and
when Z is N, X and Y are C.

In a further embodiment of the chelating composition above, Compound (I) is prepared by polymerizing Compound (Ia)

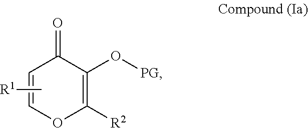

wherein
R¹ is independently selected from the group consisting of H, alkyl and alkyl optionally substituted with one of more of O, N or S;
R² is independently selected from the group consisting of H, alkyl and alkyl optionally substituted with one of more of O, N or S; and
PG is a protecting group,
with Compound (Ib)

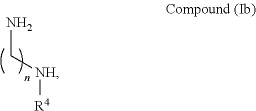

wherein
n is 1 to 12; and
R⁴ is COCCH₂R³ or a protecting group,
followed by:
when R⁴ is COCCH₂R³, removing PG to yield Compound (I); or
when R⁴ is a protecting group, removing R⁴, reacting with a suitable acrylate source, and
removing PG to yield Compound (I).

In a further embodiment of the chelating composition above,
R¹ is H;
R² is methyl;
R³ is methyl; and
n is 1 to 6 or optionally n is at least 2.

In a further embodiment of the chelating composition above, the first monomer unit is represented by Compound (II)

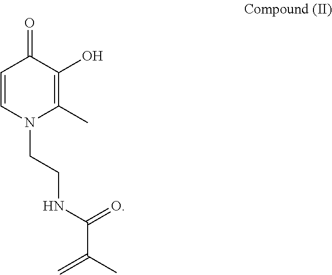

In a further embodiment of the chelating composition above, the second monomer unit is 1-vinyl-2-pyrrolidone.

In a further embodiment of the chelating composition above, the second monomer unit is N,N-dimethyl-acrylamide.

In a further embodiment of the chelating composition above, the suitable addition-fragmentation chain transfer agent is independently selected from the group consisting of 2-ethoxythiocarbonylsulfanyl-propionic acid ethyl ester and 2-ethoxythiocarbonylsulfanyl-2-methyl-propionic acid.

In a further embodiment of the chelating composition above, a residue of the addition-fragmentation chain transfer agent is removed in whole or in part from the chelating composition after polymerization.

In a further embodiment of the chelating composition above, the living animal cell prior to the addition of the composition is affected by exposure to excessive, above normal, amounts of iron in either or both of the cell's extracellular or intracellular environments and the excessive iron is contributing to chemical damage to the cell and disease in the animal and, the composition when administered, in part lowers the excessive iron levels that are contributing to chemical damage of the cell, thereby lessening damage to the cell of the animal.

In a further embodiment of the chelating composition above, said chelating composition has a molecular weight range defined by a lower molecular weight limit, as measured prior to the binding of iron of about 1500 Daltons so as not to be normally taken up into the intra-cellular aspects internal to a cell membrane of the living animal cell, and of a higher molecular weight limit sufficiently low so as to allow the composition to remain soluble in an aqueous medium.

In a further embodiment of the chelating composition above, the chelating composition comprises metal binding chemical groups of 3-hydroxy-pyridin-4-one incorporated into the carrier material comprised of pyrrolidone, acrylamide, imidazole or styrene.

In a further embodiment of the chelating composition above, the metal chelating composition is for use in treating a disease by administration into a human or other animal, that has a disease attributable to a cell or cells, or the activity of the cell or cells and, as a result of using the composition, the external and the internal aspects of the cell membrane and the internal aspects of the cell or cells underlying the cell membrane of the cell or cells are protected from chemically mediated damage as caused in whole or in part by excess of normal amounts of iron in either the external environment or internal to the cell membrane of the cell or cells.

In a further embodiment of the chelating composition above, the metal chelating composition is for use in treating a human disease which is one or more of Systemic Lupus Erythematosus Associated Nephritis; Rheumatoid Arthritis; Parkinson's Disease; Alzheimer's Disease; Fredreich's Ataxia; Amyotrophic Lateral Sclerosis; Fanconi's and related kidney disease; Type 2 Diabetes Mellitus; Hemochromatosis; Thalassemia; Macular Degeneration Eye Disease; Cardiovascular Disease or, another autoimmune, metabolic, inflammatory or neurological disease of a human or a disease of other animals where pathogenesis of the disease is in part related to higher than normal amounts of intracellular and/or extracellular iron that is contributing to pathogenesis of the disease.

In a further embodiment of the chelating composition above, the metal chelating composition is for administration in conjunction with a second iron chelator of molecular weight about <1500 Da and where the second iron chelator is normally taken up into the cell or cells and causes damage to the cell or cells as related to the nature in which it mobilizes and binds iron and where the combined activity of the chelating composition with the second iron chelator lessens injury to the cells related at least in part to excess reactive iron within the cell or cells or within the external environment of the cells.

In one embodiment, the present disclosure provides for a pharmaceutical composition comprising a chelating composition as described above and a pharmaceutically acceptable carrier, excipient or diluent and where the amount of the composition and the frequency and route of the administration of the pharmaceutical composition are adjusted to take into account the particular disease being treated such that the pharmaceutical composition addresses the relative amounts of excess, above normal, iron to be addressed for the particular disease being treated.

In a further embodiment of the pharmaceutical composition above, the pharmaceutical composition contains a second iron chelator of molecular weight about <1500 Da selected from deferoxamine, deferasirox, deferiprone, SP-420, FBS701 or another iron chelator of molecular weight about <1500 Da and where the chelating composition reduces cell or tissue damage attributable to the second chelator and therefore improves the overall efficacy of treating the disease.

In one embodiment, the present disclosure provides for a use of an iron-chelating polymer for treatment of a subject having a disease with an iron-mediated pathology, or for the manufacture of a medicament for treatment of a subject having a disease with an iron-mediated pathology, wherein:
the iron-chelating polymer comprises a reaction product of:
a first monomer unit and a second monomer units polymerized by a reversible addition-fragmentation chain transfer mechanism with the use of a suitable addition-fragmentation chain transfer agent;
wherein the first monomer unit is represented by Compound (I):

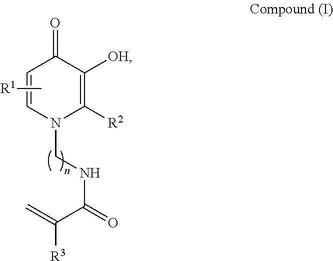

wherein:
R¹ is independently selected from the group consisting of H, alkyl optionally substituted with one of more of O, N or S;
R² is independently selected from the group consisting of H, alkyl optionally substituted with one of more of O, N or S;
R³ is independently selected from the group consisting of H, alkyl optionally substituted with one of more of O, N or S; and
n is 1 to 12;
wherein the second monomer unit is independently selected from the group consisting of 1-vinyl-2-pyrrolidone, acrylic acid, methyl methacrylate, N,N-dimethyl-acrylamide, ethyl methacrylate, N-vinyl imidazole and styrene,
wherein the iron-chelating polymer is dissolved in an aqueous medium,
wherein the iron-chelating polymer has a molecular weight, prior to chelation, of at least about 1500 Da, and
wherein the iron-chelating polymer comprises one or more intramolecular hexadentate ligands for chelating iron.

In one embodiment, the present disclosure provides for an iron-chelating polymer for use in the treatment of a subject having a disease with an iron-mediated pathology, wherein: the iron-chelating polymer comprises a reaction product of:
a first monomer unit and a second monomer units polymerized by a reversible addition-fragmentation chain transfer mechanism with the use of a suitable addition-fragmentation chain transfer agent;
wherein the first monomer unit is represented by Compound (I):

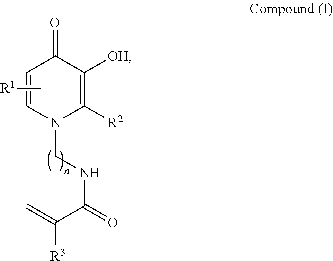

wherein:
R¹ is independently selected from the group consisting of H, alkyl and alkyl optionally substituted with one of more of O, N or S;
R² is independently selected from the group consisting of H, alkyl and alkyl optionally substituted with one of more of O, N or S;
R³ is independently selected from the group consisting of H, alkyl and alkyl optionally substituted with one of more of O, N or S; and
n is 1 to 12;
wherein the second monomer unit is independently selected from the group consisting of 1-vinyl-2-pyrrolidone, acrylic acid, methyl methacrylate, N,N-dimethyl-acrylamide, ethyl methacrylate, N-vinyl imidazole and styrene,
wherein the iron-chelating polymer is dissolved in an aqueous medium,
wherein the iron-chelating polymer has a molecular weight, prior to chelation, of at least about 1500 Da, and
wherein the iron-chelating polymer comprises one or more intramolecular hexadentate ligands for chelating iron.

In a further embodiment of the use or uses above, or the iron-chelating polymer or iron-chelating polymers for use above, the one or more suitable metal binding chemical groups are a hydroxypyridinone:

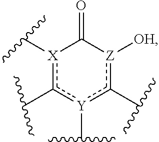

wherein X, Y and Z are independently N or C such that:
when X is N, Y and Z are C,
when Y is N, X and Z are C, and
when Z is N, X and Y are C.

In a further embodiment of the use or uses above, or the iron-chelating polymer or iron-chelating polymers for use above,
R¹ is H;
R² is methyl;
R³ is methyl; and
n is 1 to 6, optionally n is 2.

In a further embodiment of the use or uses above, or the iron-chelating polymer or iron-chelating polymers for use above, the first monomer unit is represented by Compound (II):

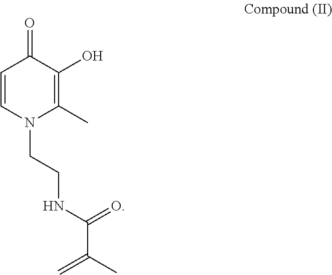

In a further embodiment of the use or uses above, or the iron-chelating polymer or iron-chelating polymers for use above, the second monomer unit is 1-vinyl-2-pyrrolidone.

In a further embodiment of the use or uses above, or the iron-chelating polymer or iron-chelating polymers for use above, the second monomer unit is N,N-dimethyl-acrylamide.

In a further embodiment of the use or uses above, or the iron-chelating polymer or iron-chelating polymers for use above, the suitable addition-fragmentation chain transfer agent is independently selected from the group consisting of 2-ethoxythiocarbonylsulfanyl-propionic acid ethyl ester and 2-ethoxythiocarbonylsulfanyl-2-methyl-propionic acid.

In a further embodiment of the use or uses above, or the iron-chelating polymer or iron-chelating polymers for use above, a residue of the addition-fragmentation chain transfer agent is removed in whole or in part from the iron-chelating polymer after polymerization.

In a further embodiment of the use or uses above, or the iron-chelating polymer or iron-chelating polymers for use above, the iron-chelating polymer lowers an extracellular concentration of free iron.

In a further embodiment of the use or uses above, or the iron-chelating polymer or iron-chelating polymers for use above, the iron-chelating polymer lowers an intracellular concentration of free iron.

In a further embodiment of the use or uses above, or the iron-chelating polymer or iron-chelating polymers for use above, the disease is an autoimmune, metabolic, inflammatory or neurological disease.

In a further embodiment of the use or uses above, or the iron-chelating polymer or iron-chelating polymers for use above, the disease is one or more of: Systemic Lupus Erythematosus Associated Nephritis, Rheumatoid Arthritis, Parkinson's Disease, Alzheimer's Disease, Fredreich's Ataxia, Amyotrophic Lateral Sclerosis, Fanconi's and related kidney disease, nephritis kidney diseases as observed with various autoimmune diseases such as Lupus Erythematosus and Rheumatoid Arthritis, Type 2 Diabetes Mellitus, Hemochromatosis, Thalassemia, Macular Degeneration Eye Disease, or Cardiovascular Disease.

In a further embodiment of the use or uses above, or the iron-chelating polymer or iron-chelating polymers for use above, iron-chelating polymer further comprises an iron chelating compound having a molecular weight of less than about 1500 Da.

In a further embodiment of the use or uses above, or the iron-chelating polymer or iron-chelating polymers for use above, the iron chelating compound is selected from deferoxamine, deferasirox, deferiprone, SP-420, FBS701, and MAHMP.

Other aspects and features of the disclosure will become apparent to those ordinarily skilled in the art upon review of the following description of specific embodiments of the disclosure.

### BRIEF DESCRIPTION OF THE DRAWINGS

Embodiments of the disclosure will now be described in greater detail with reference to the accompanying drawings, in which:

FIG. 1 shows a UV-vis spectra showing the MLCT band for iron loading of DIBI.

FIG. 2 shows the relative change in absorbance intensity at 460 nm for [Fe(MAHMP)₃]DIBI=790 μM (filled circles, concentration of Fe complex formed with MAHMP moieties in DIBI), [Fe(MAHMP)₃]=690 μM (filled squares), and [Fe(DFP)₃]-=637 μM (filled triangles) after the addition of DT with respect of corresponding control solutions in 0.1 M MOPS and pH 7.4.

FIG. 3 shows fluorescence scan figures (upper) depicting data from a representative experiment and mean fluorescence intensity values±standard error of the mean (SEM) from 3 independent experiments and plotted as bar graphs (lower); * denotes statistical significance using two-way ANOVA and the Bonferroni post-test. The data shows that DIBI reduces the intracellular labile reactive iron pool of macrophages. RAW 264.7 macrophages were loaded with 0.5 μM calcein-AM and plated into 6-well plates at 250.000 cells/well in medium alone or medium containing the indicated concentrations of DIBI or PVP. After 4, 24 and 48 h of culture, cells were harvested, and fluorescence intensity was measured by flow cytometry.

FIG. 4 shows the mean relative % ROS+SEM data from three independent experiments. FIG. 4A: RAW 264.7 macrophages were stained with 10 μM CM-H2DCFDA and cultured for 24 h with medium alone or medium containing 200 μM DIBI or 1.28 mg/mL PVP in the absence or presence of 1 μg/mL LPS. Cell fluorescence was then measured by flow cytometry. FIG. 4B: RAW 264.7 macrophages were cultured for 24 h with medium alone or medium containing 200 μM DIBI or 1.28 mg/mL PVP in the absence or presence of 100 ng/mL LPS and NO concentrations in the cell free medium were determined. Statistical significance was determined by two-way ANOVA and the Bonferroni post-test; * p<0.05. The data show that DIBI reduces macrophage synthesis of ROS and NO species.

FIG. 5 shows the mean fold expression±SEM for three independent experiments. RAW 264.7 macrophages were cultured for 6 h with medium alone or medium containing the indicated concentrations of DIBI or PVP, plus 100 ng/mL LPS. Cytokine mRNA expression relative to the medium-only control was determined by qRT-PCR. Little or no cytokine-encoding mRNAs were detected in the absence of LPS stimulation (data not shown). * denotes statistical significance using two-way ANOVA and the Bonferroni post-test. The data show that DIBI but not PVP supresses RAW 264.7 macrophage expression of IL-1β, IL-6 and IFN-β mRNA but not TNF-α mRNA.

FIG. 6 shows the mean±SEM of 4 independent experiments. RAW 264.7 macrophages that were not stimulated with LPS showed little or no cytokine production that did not change with the addition DIBI or iron (data not shown). Statistical significance was determined by ANOVA with the Tukey multiple comparisons post-test; * p<0.05. The data show that exogenous iron reverses the inhibitory effect of DIBI on macrophage synthesis of IL-6.

FIG. 7 shows UV-vis absorbance of the holotransferrin ([Holotransferrin]=47.5 μM) solutions in DFP ([DFP]=8.3 mM) and DIBI ([MAHMP]DIBI=1.2 mM) dialysis experiments showing iron(III) uptake by DIBI during initial dialysis stage using DFP as iron(III) shuttle between holotransferrin and DFP.

FIG. 8 shows the effects of DIBI on LPS-stimulated IL-6 secretion from CF15 cells; Apical (FIG. 8A) and basolateral (FIG. 8B). Upon polarization, CF15 cells were challenged with lipopolysaccharide (LPS; 200 ng/mL) and treated with DIBI (25, 50, 100, 200 μM) or media alone for 24 hours. The supernatant IL-6 amount (secreted) was normalized to total protein concentration and the data shown represent the percentage change from control level (medium only, dashed line) corresponding to average values of 526.5 pg/mL and 99.53 pg/mL IL-6 in apical and basolateral compartments, respectively. Data is presented as mean±SD, (N=3-4 independent experiments per group). *P<0.05 vs LPS. LD25: LPS+DIBI 25M; LD50: LPS+DIBI 50M; LD100: LPS+DIBI 100M; LD200: LPS+DIBI 200M.

### DETAILED DESCRIPTION

One or more illustrative embodiments have been described by way of example. It will be appreciated that embodiments and examples are provided for illustrative purposes intended for those skilled in the art, and are not meant to be limiting in any way. All references to embodiments, examples, aspects, formulas, compounds, compositions, solutions, kits and the like is intended to be illustrative and non-limiting.

A commonality to the otherwise diverse diseases set out in Table 3 may be above normal levels of labile reactive (free) iron. At least part of the pathology of these diseases with iron dysregulation may be due to incompletely coordinated iron, with full iron coordination being hexadentate in nature. This excess iron, if non-chaperoned, can be chemically labile and can be reactive within cells and also reactive in the immediate environment of cells of the disease affected tissue. In this regard, the labile reactive iron may be an underlying common feature of many otherwise diverse diseases and that these above normal amounts of reactive iron can contribute to the creation of above normal and otherwise safe levels of Reactive Oxygen Species (ROS) which in turn can cause cell and tissue damage though oxidative stress and cell damage from chemical oxidation reactions. This damage from ROS may explain at least part of the pathology of these diseases. Said diseases are referred to herein as diseases having an iron-mediated pathology. A non-exhaustive list of diseases having an iron-mediated pathology are also provided in Tables 2 and 3.

Iron requires hexadentate coordination so as to be fully stable chemically and therefore essentially non-reactive chemically, for example for ROS generation. Use of chelating molecules that do not provide full hexadentate coordination of iron as bound by a single chelator molecule may cause issues. With regards to deferoxamine, at least a portion of the iron bound by the deferoxamine starch conjugate composition may not be fully coordinated and therefore chemically reactive and chemically available for ROS formation. Such deferoxamine starch conjugate compositions could provide incompletely chaperoned iron given the nature of the starch carrier used i.e., itself, without deferoxamine, could bind iron relatively weakly in a reactive form.

Some chelators known in the art, such as deferiprone, may require 3 molecules of the chelator to fully coordinate just one iron atom and depending on the concentrations of deferiprone applied and the iron concentration in the compartments of the body that deferiprone reaches for treatment, partially coordinated iron could be expected and this incompletely coordinated iron could in turn react chemically and then generate cell/tissue damaging ROS radicals, i.e., within or around the cells and tissues exposed to the chelator. This ROS reactivity of incompletely deferiprone-coordinated iron been demonstrated to occur in vitro under conditions that could occur within cells of the body (Devanur, et al, 2008).

It can be appreciated from Table 5 that the current clinically used iron chelators deferasirox and deferiprone as well as the proposed SP-420 chelator each require either 2 or 3 chelator molecules to fully satisfy just one iron atom for tris-bidentate coordination, i.e., so as to ensure the bound iron atom is not available to participate in other chemical reactions that could for example generate cell damaging ROS. This may, at least partially, explain the significant toxicities as have been reported for these various chelators as summarized in Table 5.

Excess, i.e., above normal levels, of ROS production within or around tissue cells may be at least in part related to the pathogenesis of those various otherwise diverse non-infection/non-cancer diseases as listed as examples in Table 3. These diseases in common, include an aspect of dysregulated iron metabolism, wherein above normal amounts of potentially chemically reactive iron are present to some degree and this excess iron may lead to excess ROS production and from this in turn, cell and tissue injury, i.e., as observed in the pathogenesis of these diseases.

Use of an iron chelator that can bind iron in a non-fully coordinated and therefore ROS-reactive manner could in part generate ROS cell and tissue-damaging chemical species if a human being or animal is being treated with such a ROS-promoting iron chelator. Thus, if the human or other animal were already experiencing iron-related ROS cell and tissue damage as part of the disease pathogenesis, i.e., for those diseases linked to excess, above normal reactive iron, the treatment of these diseases with a chelator that could itself bind iron incompletely and in a ROS-promoting condition could create additional problems of ROS damage and toxicity. This problem would severely limit the use of any iron chelator that does not substantially fully coordinate iron with/upon a single chelator molecule, i.e., so as to leave the iron substantially unavailable for promoting ROS generation. An ideal chelator for treating these diseases would be one that fully coordinates iron on or within a single molecule, for example via one or more intramolecular ligands.

A physiologically normal amount of iron-mediated ROS inside cells of the human body may be important for normal cell functioning especially as related to cells involved in defence against invading microorganisms. For example, defensive macrophages ingest invading bacteria and kill the invaders within a sac-like structure of the macrophage called a phagosome, in part by applying normal, desired and useful amounts of ROS as generated within the phagosome of the macrophage, this ROS beneficially injuring and killing the invading microorganism that was engulfed by the macrophage. The ROS generated within a phagosome is also spatially separated or compartmentalized by the phagosome membrane from other intracellular aspects that could suffer ROS damage. It would therefore be undesirable to totally supress ROS production in for example, defensive cells such as macrophages while at the same time, excessive ROS production, i.e., above normal required amounts, could lead to both cell and tissue damage in the host such as is seen in the cells and tissues of the above listed diverse diseases. In this regard, macrophages may also play important roles in the above listed diseases. Macrophages play various important roles in both iron metabolism and its homeostatic regulation and also with host defence to invaders as well as with cleanup and repair of damaged cell and tissue (Sukhbaatar and Weichhart, 2018).

Iron chelators for treating diseases with an iron-mediated pathology, would be a water soluble chelator that can reach the immediate extracellular environment of the cells of the human tissues and cells of an animal with the disease being treated but of a molecular size sufficiently large so as to not normally be taken up into the internal aspects of the cells. Such a polymer may lower amounts of reactive iron primarily in the immediate environment of the cells sufficiently, for example with macrophages, to supress an excess production of cell and tissue damaging ROS while still ensuring adequate amounts of normal ROS production as needed within cells. In this regard, the lowering of excess, above normal amounts, of extracellular reactive iron with an iron chelating polymer in the extracellular environment of the cells may make iron less available for uptake by the cells being treated. This may then lower excess intracellular amounts of reactive iron as taken up from the cell's extracellular environment and consequently may reduce excess amounts of damaging ROS while not substantially affecting desired and needed amounts of ROS production, for example that ROS needed for killing of invading microbes in macrophages.

The nature of the carrier material of a chelating polymer and its selection may have substantial importance so as to avoid weakly bound and therefore reactive iron which could then promote ROS and cell damage. An ideal carrier material for a chelating polymer would support addition of chelating groups without itself binding iron in a non-chaperoned form and without interfering with the iron binding of the added chelating groups as affixed to or incorporated onto or into the carrier material.

In various embodiments, iron chelating polymers are provided that are more readily capable to fully satisfy complete iron chemical coordination, i.e., comprise one or more hexadentate ligands, and are water soluble and of a sufficiently large molecular size, so as to not to be normally taken up into cells of the body. These polymers may lower excess intracellular and/or extracellular reactive iron concentrations that are otherwise contributing to excess, i.e., above normal amounts of iron-related ROS production. The reduction of excess ROS production (i.e., above needed normal amounts) may then reduce cell and tissue damage caused by excess ROS for the disease being treated with the polymer.

The polymers described herein are useful for treatment of diseases of humans or other animals where an excess, i.e., above normal amounts of chemically reactive iron in the environment of, or within human or other animal cells, contributes to excess, i.e., above normal amounts of ROS caused by the excess reactive iron and this excess ROS causes cell and tissue damage. The diseases for treatment, include, but not limited to, various autoimmune, neurological, metabolic and inflammatory diseases.

Iron-chelating polymers for treatment of diseases with an iron-mediated pathology are disclosed herein. The polymers comprise a reaction product of: a first monomer unit and a second monomer units polymerized by a reversible addition-fragmentation chain transfer mechanism with the use of a suitable addition-fragmentation chain transfer agent.

The first monomer unit is represented by Compound (I)

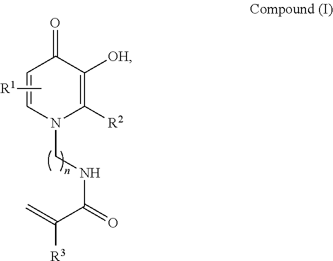

wherein
R¹ is independently selected from the group consisting of H, alkyl and alkyl optionally substituted with one or more of O, N or S;
R² is independently selected from the group consisting of H, alkyl and alkyl optionally substituted with one or more of O, N or S;
R³ is independently selected from the group consisting of H, alkyl and alkyl optionally substituted with one or more of O, N or S; and
n is 1 to 12;
and, the second monomer unit is independently selected from the group consisting of 1-vinyl-2-pyrrolidone, acrylic acid, methyl methacrylate, N,N-dimethyl-acrylamide, ethyl methacrylate, N-vinyl imidazole and styrene.

The iron-chelating polymer is water soluble. The iron-chelating polymer has a molecular weight, prior to chelation, of at least about 1500 Da. The iron-chelating polymer comprises one or more intramolecular hexadentate ligands for chelating iron, such as 3 ligands. In such embodiments, one polymer may bind 3 free iron ions.

In yet a further embodiment, the polymer may be prepared by a reversible addition-fragmentation chain transfer polymerization mechanism with the use of a suitable addition-fragmentation chain transfer agent and where a first monomer unit comprises one or more suitable hydroxypyridinone metal binding chemical groups of formula:

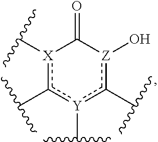

wherein X, Y and Z are independently N or C such that:
when X is N, Y and Z are C,
when Y is N, X and Z are C, and
when Z is N, X and Y are C.

In yet a further embodiment, the polymer may be prepared from a first monomer unit represented by Compound (II) and a second monomer unit comprising either 1-vinyl-2-pyrrolidone or N,N-dimethyl-acrylamide.

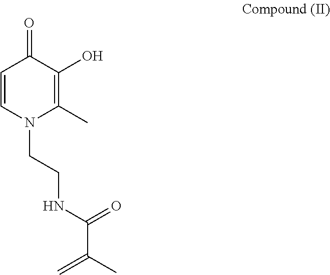

In a further embodiment, the iron-chelating polymer has a minimum molecular weight of about >1500 Da, so as not to be normally taken up into the intra-cellular aspects internal to the cell membrane of a living animal cell and is able to bind iron with up to full chemical coordination of the bound iron on, or within, a single molecule of the polymer. The iron-chelating polymer may have an upper molecular weight limit small enough so as to remain soluble in aqueous solution with its bound iron.

In a further embodiment, the metal iron-chelating polymer is used for treating a disease in a human or other animal, that has a disease attributable to a cell or cells, or the activity of a cell or cells and, the polymer binds excess, above normal, amounts of reactive iron and as a result of using the polymer, the external and the internal aspects of the cell membrane and the internal aspects of the cell underlying the cell membrane of the living cell are protected from chemically mediated damage as caused in whole or in part by an excess of normal amounts of chemically reactive iron in either the external environment or internal to the cell membrane of the cell or cells. In this regard, the excess of chemically reactive iron is contributing to excess, i.e., above normal amounts of ROS and this excess ROS is causing damage to cells or tissues as part of a disease of humans or another animal.

In yet a further embodiment, disease with an iron-mediated pathology being treated by administration of the iron-chelating polymer may be one or more of Systemic Lupus Erythematosus including associated nephritis; Rheumatoid Arthritis; Parkinson's Disease; Alzheimer's Disease; Fredreich's Ataxia; Amyotrophic Lateral Sclerosis; Fanconi's and related kidney disease; Type 2 Diabetes Mellitus; Hemochromatosis; Thalassemia; Macular Degeneration Eye Disease; Cardiovascular Disease or, another autoimmune, metabolic, inflammatory or neurological disease of a human or other animal disease where the pathogenesis of the disease is in part related to higher than normal amounts of intracellular and/or extracellular iron that is contributing to pathogenesis of the disease.

In a further preferred embodiment, the iron-chelating polymer binds iron and remains substantially soluble with its bound iron substantially in the external cellular environment of a living animal cell thereby reducing uptake of iron into the intra-cellular aspects internal to the cell membrane of the cell and as a result, the external and the internal aspects of the cell membrane and the internal aspects of the cell underlying the cell membrane of the living animal cell are, in part protected from chemically mediated damage as caused in whole or in part by excess, i.e., above normal, amounts of iron and iron related Reactive Oxygen Species (ROS) in either the external environment or internal to the cell membrane of the animal cell.

In a further embodiment, the iron-chelating polymer may be part of a pharmaceutical composition comprising the composition and a pharmaceutically acceptable carrier, excipient or diluent and where the amount of the pharmaceutical composition administered and the frequency and the route of the administration of the pharmaceutical composition are adjusted to take into account the particular disease being treated such that the pharmaceutical composition addresses the relative amounts of excess, above normal, iron to be addressed for the particular disease being treated.

In yet a further embodiment, the polymers do not excessively block normal amounts of needed iron and iron-related ROS activity, i.e., as needed for normal cell functioning and body defense but rather the polymers reduce excesses, i.e., above normal and damaging amounts of iron and as a result reduce excess, above normal amounts, of iron-related ROS.

In still a further embodiment, the polymers are for use along with, or in combination with, other known iron chelating compounds of a molecular size about <1500 Da. As discussed herein, such compounds may suffer from some degree of toxicity to animal cells or tissues due to at least in part, their molecular size and their binding of incompletely coordinated iron which can lead to iron related toxicity reactions. In this regard, the incident polymers may help to, in part, overcome the toxicity limitations of the other known chelating agents, thus providing an improved overall iron related treatment efficacy of the combination and with reduced limitations due to toxicity. For example, deferoxamine, deferasirox, deferiprone, SP-420, FBS701, MAHMP and other suitable compounds known in the art.

### PATENT REFERENCES CITED

Holbein et al, U.S. Pat. No. 10,709,784. Metal chelating compositions and methods for controlling the growth or activities of a living cell or organism.
Holbein et al, U.S. Pat. No. 11,059,785. Polymeric metal chelating compositions and methods of preparing same for controlling growth and activities of living cells.
Liu et al, U.S. Pat. No. 8,334,320. 2010. Methods for chelation therapy.

### NON-PATENT REFERENCES CITED

Aali, M., Caldwell, A., Li, A., Holbein, B., Chappe, V., & Lehmann, C. (2020). DIBI, a novel polymeric iron chelator modulates IL-6 and IL-8 secretion from Cystic Fibrosis airway epithelial cells in response to endotoxin induction. Journal of Cellular Biotechnology 6: 161-170. https://content.iospress.com/articles/journal-of-cellular-biotechnology/jcb209011
Akinc, A.; Chan-Daniels, A.; Sehgal, A.; Foster, D.; Bettencourt, B. R.; Hettinger, J.; Racie, T.; Aubin, J.; Kuchimanchi, S.; Epstein-Barash, H.; et al. Targeting the Hepcidin Pathway with RNAi Therapeutics for the Treatment of Anemia. Blood 2011, 118, 688, doi:10.1182/blood.v118.21.688.688
Ali, M. K.; Kim, R. Y.; Brown, A. C.; Donovan, C.; Vanka, K. S.; Mayall, J. R.; Liu, G.; Pillar, A. L.; Jones-Freeman, B.; Xenaki, D.; et al. Critical role for iron accumulation in the pathogenesis of fibrotic lung disease. J. Pathol. 2020, 251, 49-62, doi:10.1002/path.5401
Alfonso-Prieto, M.; Biarnés, X.; Vidossich, P.; Rovira, C. The molecular mechanism of the catalase reaction. J. Am. Chem. Soc. 2009, 131, 11751-11761, doi:10.1021/ja9018572
Andreini, C.; Putignano, V.; Rosato, A.; Banci, L. The human iron-proteome. Metallomics 2018, 10, 1223-1231, doi:10.1039/c8mt00146d
Ang, M. T. C., Gumbau-Brisa, R., Allan, D. S., McDonald, R., Ferguson, M. J., b Holbein, B. E., & Bierenstiel, M. (2018). DIBI, a 3-hydroxypyridin-4-one chelator iron-binding polymer with enhanced antimicrobial activity. MedChemComm, 9, 1206-1212. https://doi.org/10.1039/c8md00192h
Angeli, J. P. F., Shah, R., Pratt, D. A. and Conrad, M., 2017. Ferroptosis inhibition: mechanisms and opportunities. Trends in pharmacological sciences, 38(5), pp. 489-
Badeli, H., et al. (2019). Early Kidney Damage Markers after Deferasirox Treatment in Patients with Thalassemia Major: A Case-Control Study. Oxidative Medicine and Cellular Longevity, 2019, 5461617. https://doi.org/10.1155/2019/5461617
Baker, J. F.; Ghio, A. J. Iron homoeostasis in rheumatic disease. Rheumatology (Oxford). 2009, 48, 1339-1344, doi:10.1093/rheumatology/kep221
Bao, W.; Rong, Y.; Rong, S.; Liu, L. Dietary iron intake, body iron stores, and the risk of type 2 diabetes: A systematic review and meta-analysis. BMC Med. 2012, 10, 1-13, doi:10.1186/1741-7015-10-119
Beinert, H.; Kennedy, M. C.; Stout, C. D. Aconitase as iron-sulfur protein, enzyme, and iron-regulatory protein. Chem. Rev. 1996, 96, 2335-2373, doi:10.1021/cr950040z
Bergeron, Raymond J., et al. (2014). “Desferrithiocin: A search for clinically effective iron chelators.” Journal of medicinal chemistry 57.22 9259-9291 https://pubs.acs.org/doi/abs/10.1021/jm500828f
Bullen, J.; Griffiths, E.; Rogers, H.; Ward, G. Sepsis: The critical role of iron. Microbes Infect. 2000, 2, 409-415, doi:10.1016/S1286-4579(00)00326-9
Cabantchik, Z. I.; Breuer, W.; Zanninelli, G.; Cianciulli, P. LPI-labile plasma iron in iron overload. Best Pract. Res. Clin. Haematol. 2005, 18, 277-287, doi:10.1016/j.beha.2004.10.003.
Cabantchik, Z. I. Labile iron in cells and body fluids: Physiology, pathology, and pharmacology. Front. Pharmacol. 2014, 5, 1-11, doi:10.3389/fphar.2014.00045
Candelaria, P. V.; Leoh, L. S.; Penichet, M. L.; Daniels-Wells, T. R. Antibodies Targeting the Transferrin Receptor 1 (TfR1) as Direct Anti-cancer Agents. Front. Immunol. 2021, 12
Casu, C., Nemeth, E. and Rivella, S., 2018. Hepcidin agonists as therapeutic tools. Blood, The Journal of the American Society of Hematology, 131(16), pp. 1790-1794
Chang, H. C.; Bayeva, M.; Taiwo, B.; Palella, F. J.; Hope, T. J.; Ardehali, H. Short communication: High cellular iron levels are associated with increased HIV infection and replication. AIDS Res. Hum. Retroviruses 2015, 31, 305-312, doi:10.1089/aid.2014.0169
Chen, H., 2022. Iron metabolism in nonalcoholic fatty liver disease: a promising therapeutic target. Liver Research
Chowers, I.; Wong, R.; Dentchev, T.; Farkas, R. H.; Iacovelli, J.; Gunatilaka, T. L.; Medeiros, N. E.; Presley, J. B.; Campochiaro, P. A.; Curcio, C. A.; et al. The iron carrier transferrin is upregulated in retinas from patients with age-related macular degeneration. Investig. Ophthalmol. Vis. Sci. 2006, 47, 2135-40, doi:10.1167/iovs.05-1135
Clark, M. A.; Goheen, M. M.; Cerami, C. Influence of host iron status on Plasmodium falciparum infection. Front. Pharmacol. 2014, 5, 1-12, doi:10.3389/fphar.2014.00084
Clemens, D. L.; Horwitz, M. A. The Mycobacterium tuberculosis phagosome interacts with early endosomes and is accessible to exogenously administered transferrin. J. Exp. Med. 1996, 184, 1349-1355, doi:10.1084/jem.184.4.1349
Cooksey, Robert C., et al. (2010). Dietary iron restriction or iron chelation protects from diabetes and loss of β-cell function in the obese (ob/ob lep−/−) mouse.” American Journal of Physiology-Endocrinology and Metabolism 298.6: E1236-E1243; https://journals.physiology.org/doi/full/10.1152/ajpendo.00022.2010)
Cronin, S. J. F.; Woolf, C. J.; Weiss, G.; Penninger, J. M. The Role of Iron Regulation in Immunometabolism and Immune-Related Disease. Front. Mol. Biosci. 2019, 6, 1-19, doi:10.3389/fmolb.2019.00116
Das, N.; Raymick, J.; Sarkar, S. Role of metals in Alzheimer's disease. Metab. Brain Dis. 2021, 36, 1627-1639, doi:10.1007/s11011-021-00765-w
David, S.; Jhelum, P.; Ryan, F.; Jeong, S. Y.; Kroner, A. Dysregulation of Iron Homeostasis in the Central Nervous System and the Role of Ferroptosis in Neurodegenerative Disorders. Antioxidants Redox Signal. 2022, 37, 150-170, doi:10.1089/ars.2021.0218
Devanur, Lakshmi D., Hendrik Neubert, and Robert C. Hider. 2008. The Fenton activity of iron (III) in the presence of deferiprone. Journal of pharmaceutical sciences 97.4 (2008): 1454-1467; https://www.sciencedirect.com/science/article/abs/pii/S0022354916325473).
Ezraty, B.; Barras, F. The “liaisons dangereuses” between iron and antibiotics. FEMS Microbiol. Rev. 2016, 40, 418-435, doi:10.1093/femsre/fuw004
Fang, F. C. Antimicrobial reactive oxygen and nitrogen species: Concepts and controversies. Nat. Rev. Microbiol. 2004, 2, 820-832, doi:10.1038/nrmicro1004
Fang, X.; Ardehali, H.; Min, J.; Wang, F. The molecular and metabolic landscape of iron and ferroptosis in cardiovascular disease. Nat. Rev. Cardiol. 2023, 20, 7-23, doi:10.1038/s41569-022-00735-4
Fang, X.; Wang, H.; Han, D.; Xie, E.; Yang, X.; Wei, J.; Gu, S.; Gao, F.; Zhu, N.; Yin, X.; et al. Ferroptosis as a target for protection against cardiomyopathy. Proc. Natl. Acad. Sci. U.S.A. 2019, 116, 2672-2680, doi:10.1073/pnas.1821022116
Fernindez-Real, J. M.; López-Bermejo, A.; Ricart, W. Cross-talk between iron metabolism and diabetes. Diabetes 2002, 51, 2348-2354, doi:10.2337/diabetes.51.8.2348
Fibach, E., & Dana, M. (2019). Oxidative stress in β-Thalassemia. Molecular Diagnosis & Therapy, 23(2), 245-261. https://doi.org/10.1007/s40291-018-0373-5
Fokam, D.; Dickson, K.; Kamali, K.; Holbein, B.; Colp, P.; Stueck, A.; Zhou, J.; Lehmann, C. Iron chelation in murine models of systemic inflammation induced by gram-positive and gram-negative toxins. Antibiotics 2020, 9, 1-15, doi:10.3390/antibiotics9060283
Foley, T. L., & Simeonov, A. (2012). Targeting iron assimilation to develop new antibacterials. Expert Opin Drug Discov, 7(9), 831-847. https://doi.org/10.1517/17460441.2012.708335
Giacco, F.; Brownlee, M. Oxidative stress and diabetic complications. Circ. Res. 2010, 107, 1058-1070, doi:10.1161/CIRCRESAHA.110.223545
Giardina, B. J.; Shahzad, S.; Huang, W.; Wilks, A. Heme uptake and utilization by hypervirulent Acinetobacter baumannii LAC-4 is dependent on a canonical heme oxygenase (abHemO). Arch. Biochem. Biophys. 2019, 672, 1-21, doi:10.1016/j.abb.2019.108066
Ghassemi-Rad, J., Fernando, W., Holbein, B. E., Hoskin, D. W. (2022). Iron withdrawal with DIBI, a novel 3-hydroxypyridin-4-one chelator iron-binding polymer, attenuates macrophage inflammatory responses. accepted/in press Dec. 31, 2021 for 2022 publication in Advanced Pharmaceutical Bulletin.
Greenshields, A. L.; Power Coombs, M. R.; Fernando, W.; Holbein, B. E.; Hoskin, D. W. DIBI, a novel 3-hydroxypyridin-4-one chelator iron-binding polymer, inhibits breast cancer cell growth and functions as a chemosensitizer by promoting S-phase DNA damage. BioMetals 2019, 32, 909-921, doi:10.1007/s10534-019-00222-3
Gumbau-Brisa, R., Ang, M. T. C., Holbein, B. E., & Bierenstiel, M. (2020). Enhanced Fe3+ binding through cooperativity of 3-hydroxypyridin-4-one groups within a linear co-polymer: wrapping effect leading to superior antimicrobial activity. Biometals: an international journal on the role of metal ions in biology, biochemistry, and medicine, 33(6), 339-351. https://doi.org/10.1007/s10534-020-00253-1
Hadzhieva, M, et al (2014). Iron metabolism and the role of iron in neurodegenerative disorders, Neuropathology and applied neurobiology 40.3 240-257, https://doi.org/10.1111/nan.12096.
Halliwell, B.; Adhikary, A.; Dingfelder, M.; Dizdaroglu, M. Hydroxyl radical is a significant player in oxidative DNA damage: In vivo. Chem. Soc. Rev. 2021, 50, 8355-8360, doi:10.1039/d1cs00044f
Hanstock, H. G.; Edwards, J. P.; Walsh, N. P. Tear lactoferrin and lysozyme as clinically relevant biomarkers of mucosal immune competence. Front. Immunol. 2019, 10, 1-11, doi:10.3389/fimmu.2019.01178
Harrison, A. V.; Lorenzo, F. R.; McClain, D. A. Iron and the Pathophysiology of Diabetes. Annu. Rev. Physiol. 2023, 85, 339-62, doi:10.1146/annurev-physiol-022522-102832
Hayyan, M.; Hashim, M. A.; Alnashef, I. M. Superoxide Ion: Generation and Chemical Implications. Chem. Rev. 2016, 116, 3029-3085, doi:10.1021/acs.chemrev.5b00407
Hider, Robert C., et al. (2015). Deferitazole, a new orally active iron chelator.” Dalton Transactions 44.11 5197-5204; https://pubs.rsc.org/en/content/articlelanding/2015/dt/c5dt00063g/unauth
Hissen, A. H. T.; Chow, J. M. T.; Pinto, L. J.; Moore, M. M. Survival of Aspergillus fumigatus in Serum Involves Removal of Iron from Transferrin: The Role of Siderophores. Infect. Immun. 2004, 72, 1402-1408, doi:10.1128/IAI.72.3.1402-1408.2004
Holbein, B. E. Enhancement of Neisseria meningitidis infection in mice by addition of iron bound to transferrin. Infect. Immun. 1981, 34, 120-125, doi:10.1128/iai.34.1.120-125.1981
Holbein B. E, et al. 2021. Iron dependence, the Achilles' heel of antibiotic resistant microbes: iron withdrawing anti-infectives for new host-directed therapies: a review. Environmental Chemistry Letters 19, 2789-2808; https://doi.org/10.1007/s10311-021-01242-7
Ibrahim, A. S., et al. (2010). The iron chelator deferasirox enhances liposomal amphotericin B efficacy in treating murine invasive pulmonary aspergillosis. Journal of Antimicrobial Chemotherapy, 65(2), 289-292. https://doi.org/10.1093/jac/dkp426
Ismaiel, A.; Srouji, N. Al Anemia of Chronic Disease: Epidemiology and Pathophysiological Mechanisms—Literature Review. Glob. J. Med. Ther. 2020, 2, 8-16, doi:10.46982/gjmt.2020.109
Jordan, A.; Reichard, P. Ribonucleotide reductases. Annu. Rev. Biochem. 1998, 67, 71-98, doi:10.1146/annurev.biochem.75.103004.142443
Joshi, P. K., Patel, S. C., Shreya, D., Zamora, D. I., Patel, G. S., Grossmann, I., Rodriguez, K., Soni, M. and Sange, I. (2021). Hereditary Hemochromatosis: A Cardiac Perspective. Cureus, 13(11).
Kakhlon, O.; Cabantchik, Z. I. The labile iron pool: Characterization, measurement, and participation in cellular processes. Free Radic. Biol. Med. 2002, 33, 1037-1046, doi:10.1016/S0891-5849(02)01006-7
Kakizaki, S.; Takagi, H.; Horiguchi, N.; Toyoda, M.; Takayama, H.; Nagamine, T.; Mori, M.; Kato, N. Iron enhances hepatitis C virus replication in cultured human hepatocytes. Liver 2000, 20, 125-128, doi:10.1034/j.1600-0676.2000.020002125.x
Kemp, J. D., Cardillo, T., Stewart, B. C., Kehrberg, E., Weiner, G., Hedlund, B., & Naumann, P. W. (1995). Inhibition of lymphoma growth in vivo by combined treatment with hydroxyethyl starch deferoxamine conjugate and IgG monoclonal antibodies against the transferrin receptor. Cancer research, 55(17), 3817-3824.
Knight, S. A. B.; Vilaire, G.; Lesuisse, E.; Dancis, A. Iron acquisition from transferrin by Candida albicans depends on the reductive pathway. Infect. Immun. 2005, 73, 5482-5492Liu, doi:10.1128/IAI.73.9.5482-5492.2005
Kochi, M.; Kohagura, K.; Shiohira, Y.; Iseki, K.; Ohya, Y. Chronic kidney disease, inflammation, and cardiovascular disease risk in rheumatoid arthritis. J. Cardiol. 2018, 71, 277-283, doi:10.1016/j.jjcc.2017.08.008
Lalonde, R. G.; Holbein, B. E. Role of iron in Trypanosoma cruzi infection of mice. J. Clin. Invest. 1984, 73, 470-476, doi:10.1172/JCI111233
Lan, P.; Pan, K. han; Wang, S. jia; Shi, Q. cheng; Yu, Y. xian; Fu, Y.; Chen, Y.; Jiang, Y.; Hua, X. ting; Zhou, J. Cang; et al. High Serum Iron level is Associated with Increased Mortality in Patients with Sepsis. Sci. Rep. 2018, 8, 1-7, doi:10.1038/s41598-018-29353-2
Lehmann, C.; Islam, S.; Jarosch, S.; Zhou, J.; Hoskin, D.; Greenshields, A.; Al-Banna, N.; Sharawy, N.; Sczcesniak, A.; Kelly, M.; et al. The utility of iron chelators in the management of inflammatory disorders. Mediators Inflamm. 2015, 2015, 1-12, doi:10.1155/2015/516740
Lei, P.; Bai, T.; Sun, Y. Mechanisms of ferroptosis and relations with regulated cell death: A review. Front. Physiol. 2019, 10, 1-13, doi:10.3389/fphys.2019.00139
Li, D.; Jiang, C.; Mei, G.; Zhao, Y.; Chen, L.; Liu, J.; Tang, Y.; Gao, C.; Yao, P. Quercetin alleviates ferroptosis of pancreatic R cells in type 2 diabetes. Nutrients 2020, 12, doi:10.3390/nu12102954
Li, H.; Steyger, P. Synergistic ototoxicity due to noise exposure and aminoglycoside antibiotics. Noise Heal. 2009, 11, 26-32, doi:10.4103/1463-1741.45310
Li, J.; Cao, F.; Yin, H. Liang; Huang, Z. Jian; Lin, Z. Tao; Mao, N.; Sun, B.; Wang, G. Ferroptosis: past, present and future. Cell Death Dis. 2020, 11, 1-13, doi:10.1038/s41419-020-2298-2
Li S and Zhang X (2021). Iron in Cardiovascular Disease: Challenges and Potentials. Front. Cardiovasc. Med. 8:707138, https://www.ncbi.nlm.nih.gov/pmc/articles/PMC8669346/
Liu J-L et al (2018). Iron and Alzheimer's Disease: From Pathogenesis to Therapeutic Implications. Front. Neurosci. 12:632. https://www.frontiersin.org/articles/10.3389/fnins.2018.00632/full
Liu, Q.; Wu, J.; Zhang, X.; Wu, X.; Zhao, Y.; Ren, J. Iron homeostasis and disorders revisited in the sepsis. Free Radic. Biol. Med. 2021, 165, 1-13, doi:10.1016/j.freeradbiomed.2021.01.025
Loh, A.; Hadziahmetovic, M.; Dunaief, J. L. Iron homeostasis and eye disease. Biochim. Biophys. Acta-Gen. Subj. 2009, 1790, 637-649, doi:10.1016/j.bbagen.2008.11.001
Mach, J.; Sutak, R. Iron in parasitic protists—from uptake to storage and where we can interfere. Metallomics 2020, 12, 1335-1347, doi:10.1039/d0mt00125b
Maniam, P., Essilfie, A T., Kalimutho, M. et al. (2021). Increased susceptibility of cystic fibrosis airway epithelial cells to ferroptosis. Biol Res 54, 38 https://doi.org/10.1186/s40659-021-00361-3
Mao, W. L.; Hu, Y.; Lou, Y. F.; Chen, Y. M.; Zhang, J. W. Abnormal serum iron markers in chronic hepatitis B virus infection may be because of liver injury. Eur. J. Gastroenterol. Hepatol. 2015, 27, 130-136, doi:10.1097/MEG.0000000000000247
McDonough, M. A.; Loenarz, C.; Chowdhury, R.; Clifton, I. J.; Schofield, C. J. Structural studies on human 2-oxoglutarate dependent oxygenases. Curr. Opin. Struct. Biol. 2010, 20, 659-672, doi:10.1016/j.sbi.2010.08.006
Medeiros, M. S.; Schumacher-Schuh, A.; Cardoso, A. M.; Bochi, G. V.; Baldissarelli, J.; Kegler, A.; Santana, D.; Chaves, C. M. M. B. S.; Schetinger, M. R. C.; Moresco, R. N.; et al. Iron and oxidative stress in Parkinson's disease: An observational study of injury biomarkers. PLoS One 2016, 11, doi:10.1371/journal.pone.0146129
Miller, C. E.; Rock, J. D.; Ridley, K. A.; Williams, P. H.; Ketley, J. M. Utilization of lactoferrin-bound and transferrin-bound iron by Campylobacter jejuni. J. Bacteriol. 2008, 190, 1900-11, doi:10.1128/JB.01761-07
Minakami, R.; Sumimoto, H. Phagocytosis-coupled activation of the superoxide-producing phagocyte oxidase, a member of the NADPH oxidase (Nox) family. Int. J. Hematol. 2006, 84, 193-198, doi:10.1532/IJH97.06133
Modun, B.; Evans, R. W.; Joannou, C. L.; Williams, P. Receptor-mediated recognition and uptake of iron from human transferrin by Staphylococcus aureus and Staphylococcus epidermidis. Infect. Immun. 1998, 66, 3591-3596, doi:10.1128/iai.66.8.3591-3596.1998
Murdoch, C. C.; Skaar, E. P. Nutritional immunity: the battle for nutrient metals at the host-pathogen interface. Nat. Rev. Microbiol. 2022, 20, 657-670, doi:10.1038/s41579-022-00745-6
Nemeth, E.; Ganz, T. Hepcidin and Iron in Health and Disease. Annu. Rev. Med. 2022, 74, 261-77, doi:10.1016/j.mam.2020.100906
Neupane, G. P., & Kim, D. M. (2010). In vitro time-kill activities of ciprofloxacin alone and in combination with the iron chelator deferasirox against Vibrio vulnificus. European Journal of Clinical Microbiology & Infectious Diseases, 29(4), 407-410. https://doi.org/10.1007/s10096-010-0875-5
Niihara, Y., Shalev, O., Hebbel, R. P., Wu, H., Tu, A., Akiyama, D. S., & Tanaka, K. R. (2000). Desferrioxamine (DFO) conjugated with starch decreases NAD redox potential of intact red blood cells (RBC): evidence for DFO as an extracellular inducer of oxidant stress in RBC. American journal of hematology, 65(4), 281-284. https://onlinelibrary.wiley.com/doi/abs/10.1002/1096-8652(200012)65:4%3C281::AID-AJH3%3E3.0.CO; 2-K
Olwal, C. O.; Nganyewo, N. N.; Tapela, K.; Djomkam Zune, A. L.; Owoicho, O.; Bediako, Y.; Duodu, S. Parallels in Sepsis and COVID-19 Conditions: Implications for Managing Severe COVID-19. Front. Immunol. 2021, 12, 1-6, doi:10.3389/fimmu.2021.602848
Ogger, P. P.; Byrne, A. J. Lung fibrosis enters the iron age. J. Pathol. 2020, 252, 1-3, doi:10.1002/path.5489
Padmanaban Venkatesan, et al (2021). Evidence of dysregulated iron homeostasis in newly diagnosed diabetics, but not in pre-diabetics, Journal of Diabetes and its Complications, Volume 35, https://doi.org/10.1016/j.jdiacomp.2021.107977
Pantopoulos, K. Inherited Disorders of Iron Overload. Front. Nutr. 2018, 5, 1-11, doi:10.3389/fnut.2018.00103
Parker, M. W.; Blake, C. C. F. Iron- and manganese-containing superoxide dismutases can be distinguished by analysis of their primary structures. FEBS Lett. 1988, 229, 377-382, doi:10.1016/0014-5793(88)81160-8
Parquet, M. d. C., et al (2018). Novel Iron-Chelator DIBI Inhibits Staphylococcus aureus Growth, Suppresses Experimental MRSA Infection in Mice and Enhances the Activities of Diverse Antibiotics in vitro. Frontiers in Microbiology, 9, 1811. https://doi.org/https://doi.org/10.3389/fmicb.2018.01811
Pfaender, S.; Grabrucker, A. M. Characterization of biometal profiles in neurological disorders. Metallomics 2014, 6, 960-977, doi:10.1039/c4mt00008k
Pretorius E, et al, (2017). Major involvement of bacterial components in rheumatoid arthritis and its accompanying oxidative stress, systemic inflammation and hypercoagulability. Experimental Biology and Medicine.; 242(4):355-373. doi:10.1177/1535370216681549)
Prinsen, B. H. C. M. T.; De Sain-van der Velden, M. G. M.; Kaysen, G. A.; Straver, H. W. H. C.; Van Rijn, H. J. M.; Stellaard, F.; Berger, R.; Rabelink, T. J. Transferrin synthesis is increased in nephrotic patients insufficiently to replace urinary losses. J. Am. Soc. Nephrol. 2001, 12, 1017-1025, doi:10.1681/asn.v1251017
Ray, P. D.; Huang, B. W.; Tsuji, Y. Reactive oxygen species (ROS) homeostasis and redox regulation in cellular signaling. Cell. Signal. 2012, 24, 981-990, doi:10.1016/j.cellsig.2012.01.008
Ren, J. X.; Sun, X.; Yan, X. L.; Guo, Z. N.; Yang, Y. Ferroptosis in Neurological Diseases. Front. Cell. Neurosci. 2020, 14
Savage, K. A., Parquet, M. C., Allan, D. S., Davidson, R. J., Holbein, B. E., Lilly, E. A., & Fidel, P. L. J. (2018). Iron Restriction to Clinical Isolates of Candida albicans by the Novel Chelator DIBI Inhibits Growth and Increases Sensitivity to Azoles In Vitro and In Vivo in a Murine Model of Experimental Vaginitis. Antimicrobial Agents and Chemotherapy, 62(8), e02576-02517. https://doi.org/10.1128/AAC.02576-17.
Schaible, U. E.; Kaufmann, S. H. E. Iron and microbial infection. Nat. Rev. Microbiol. 2004, 946-953, doi:10.1038/nrmicro1046
Schmidt, S. The role of iron in viral infections. Front. Biosci. (Landmark Ed. 2020, 25, 893-911, doi:10.7868/s2410993220030033
Schumacher-Schuh M S, et al. (2016). Iron and Oxidative Stress in Parkinson's Disease: An Observational Study of Injury Biomarkers. PLoS ONE 11(1): e0146129. https://journals.plos.org/plosone/article?id=10.1371/journal.pone.0146129
Shen, Y.; Li, X.; Dong, D.; Zhang, B.; Xue, Y.; Shang, P. Transferrin receptor 1 in cancer: a new sight for cancer therapy. Am. J. Cancer Res. 2018, 8, 916-931
Skaar, E. P.; Schneewind, O. Iron-regulated surface determinants (Isd) of Staphylococcus aureus: Stealing iron from heme. Microbes Infect. 2004, 6, 390-397, doi:10.1016/j.micinf.2003.12.008
Smith C P and F Thévenod, (2009). Iron transport and the kidney, Biochimica et Biophysica Acta (BBA)—General Subjects, Volume 1790: 724-730, https://doi.org/10.1016/j.bbagen.2008.10.010
Sukhbaatar, Nyamdelger, and Thomas Weichhart. (2018): Iron regulation: macrophages in control. Pharmaceuticals 11.4 137; https://www.mdpi.com/1424-8247/11/4/137
Swaminathan, S.; Shah, S. V. Novel approaches targeted toward oxidative stress for the treatment of chronic kidney disease. Curr. Opin. Nephrol. Hypertens. 2008, 17, 143-8, doi:10.1097/MNH.0b013e3282f4e539
Tacke, F.; Nuraldeen, R.; Koch, A.; Strathmann, K.; Hutschenreuter, G.; Trautwein, C.; Strnad, P. Iron parameters determine the prognosis of critically ill patients. Crit. Care Med. 2016, 44, doi:10.1097/CCM.0000000000001607
Taher, Ali T., et al. (2017). Safety and pharmacokinetics of the oral iron chelator SP-420 in β-thalassemia.” American journal of hematology 92.12 1356-1361; https://onlinelibrary.wiley.com/doi/full/10.1002/ajh.24914
Taher, A., Kourakli-Symeonidis, A., Tantiworawit, A., Wong, P. and Szecsody, P., 2022. S272: Safety and Preliminary Pharmacodynamic Effects of the Ferroportin Inhibitor Vamifeport (VIT-2763) in Patients with Non-Transfusion-Dependent Beta Thalassemia (NTDT): Results from a Phase 2A Study. HemaSphere, 6, pp. 173-174
Theut L R, et al (2020). Evidence of Renal Iron Accumulation in a Male Mouse Model of Lupus. Front. Med. 7:516. https://www.frontiersin.org/articles/10.3389/fmed.2020.00516/full
Thompson, M. G., et al (2012). Antibacterial Activities of Iron Chelators against Common Nosocomial Pathogens. Antimicrobial Agents and Chemotherapy, 56(10), 5419-5421. https://doi.org/10.1128/AAC.01197-12
Torti, S. V.; Manz, D.; Paul, B.; Blanchette-Farra, N.; Torti, F. M. Iron and cancer. Iron Hum. Dis. 2018, 38, 97-125, doi:10.1201/9781351073899
Torti, S. V.; Torti, F. M. Iron and cancer: More ore to be mined. Nat. Rev. Cancer 2013, 13, 342-355, doi:10.1038/nrc3495
Troxell, B.; Hassan, H. M. Transcriptional regulation by Ferric Uptake Regulator (Fur) in pathogenic bacteria. Front. Cell. Infect. Microbiol. 2013, 4, 1-13, doi:10.3389/fcimb.2013.00059
Toyokuni, S.; Yanatori, I.; Kong, Y.; Zheng, H.; Motooka, Y.; Jiang, L. Ferroptosis at the crossroads of infection, aging and cancer. Cancer Sci. 2020, 111, 2665-2671, doi:10.1111/cas.14496
Ueda, K., et al, (2018). Iron promotes oxidative cell death caused by bisretinoids of retina. Proceedings of the National Academy of Sciences, 115(19):4963-4968; https://www.pnas.org/content/115/19/4963.short)
Venkatesan, P.; Varghese, J.; Arthi, T. S.; James, J. V.; Anura, A.; Prasad, J.; Jacob, M. Evidence of dysregulated iron homeostasis in newly diagnosed diabetics, but not in pre-diabetics. J. Diabetes Complications 2021, 35, 1-8, doi:10.1016/j.jdiacomp.2021.107977
Verstovsek, S., Kuykendall, A. T., Hoffman, R., Ginzburg, Y., Pemmaraju, N., Valone, F., Modi, N. B., Khanna, S., O'Connor, P. G., Gupta, S. K. and Kiladjian, J. J., 2021. A phase 3 study of the hepcidin mimetic rusfertide (PTG-300) in patients with polycythemia vera. Blood, 138, p. 1504
Vinchi, F. Non-Transferrin-Bound Iron in the Spotlight: Novel Mechanistic Insights into the Vasculotoxic and Atherosclerotic Effect of Iron. Antioxidants Redox Signal. 2021, 35, 387-414, doi:10.1089/ars.2020.8167
Vlachodimitropoulou, E., et al. (2017). Eltrombopag: a powerful chelator of cellular or extracellular iron(III) alone or combined with a second chelator. Blood, 130(17), 1923-1933. https://doi.org/10.1182/blood-2016-10-740241
Wei, Y.; Ye, W.; Zhao, W. Serum iron levels decreased in patients with HBV-related hepatocellular carcinoma, as a risk factor for the prognosis of HBV-related HCC. Front. Physiol. 2018, 9, 1-10, doi:10.3389/fphys.2018.00066
Weiss, G.; Ganz, T.; Goodnough, L. T. Anemia of inflammation. Blood 2019, 133, 40-50, doi:10.1182/blood-2018-06-856500
Wicinski, M.; Liczner, G.; Cadelski, K.; Kolnierzak, T.; Nowaczewska, M.; Malinowski, B. Anemia of chronic diseases: Wider diagnostics—better treatment? Nutrients 2020, 12, 1-17, doi:10.3390/nu12061784
Williams, R. J. P. The mechanism of cytochrome oxidase and other reaction centres for electron/proton pumping. FEBS Lett. 1987, 226, 1-7, doi:10.1016/0014-5793(87)80539-2
Wilson, M. E.; Vorhies, R. W.; Andersen, K. A.; Britigan, B. E. Acquisition of iron from transferrin and lactoferrin by the protozoan Leishmania chagasi. Infect. Immun. 1994, 62, 3262-3269, doi:10.1128/iai.62.8.3262-3269.1994
Wincup, C.; Sawford, N.; Rahman, A. Pathological mechanisms of abnormal iron metabolism and mitochondrial dysfunction in systemic lupus erythematosus. Expert Rev. Clin. Immunol. 2021, 17, 957-967, doi:10.1080/1744666X.2021.1953981
Wu, T.; Sempos, C. T.; Freudenheim, J. L.; Muti, P.; Smit, E. Serum iron, copper and zinc concentrations and risk of cancer mortality in US adults. Ann. Epidemiol. 2004, 14, 195-201, doi:10.1016/51047-2797(03)00119-4
Xiao, R.; Kisaalita, W. S. Iron acquisition from transferrin and lactoferrin by Pseudomonas aeruginosa pyoverdin. Microbiology 1997, 143, 2509-2515, doi:10.1099/00221287-143-7-2509
Xu, T., Zhang, X., Liu, Y., Wang, H., Luo, J., Luo, Y. and An, P., 2021. Effects of dietary polyphenol supplementation on iron status and erythropoiesis: a systematic review and meta-analysis of randomized controlled trials. The American Journal of Clinical Nutrition, 114(2), pp. 780-793
Yamamoto, S.; Okujo, N.; Kataoka, H.; Narimatsu, S. Siderophore-mediated utilization of transferrin- and lactoferrin-bound iron by Acinetobacter baumannii. J. Heal. Sci. 1999, 45, 297-302, doi:10.1248/jhs.45.297
Yang, W. S.; Stockwell, B. R. Synthetic Lethal Screening Identifies Compounds Activating Iron-Dependent, Nonapoptotic Cell Death in Oncogenic-RAS-Harboring Cancer Cells. Chem. Biol. 2008, 15, 234-45, doi:10.1016/j.chembiol.2008.02.010
Yiannikourides, A.; Latunde-Dada, G. A Short Review of Iron Metabolism and Pathophysiology of Iron Disorders. Medicines 2019, 6, 1-15, doi:10.3390/medicines6030085
Zeidan, A. M., & Griffiths, E. A. (2018). To chelate or not to chelate in MDS: That is the question! Blood Reviews, 32(5), 368-377. https://doi.org/https://doi.org/10.1016/j.blre.2018.03.002
Zhang, C. Essential functions of iron-requiring proteins in DNA replication, repair and cell cycle control. Protein Cell 2014, 5, 750-760, doi:10.1007/s13238-014-0083-7
Zhang, J.; Sheng, S.; Wang, W.; Dai, J.; Zhong, Y.; Ren, J.; Jiang, K.; Li, S.; Bian, X.; Liu, L. Molecular Mechanisms of Iron Mediated Programmed Cell Death and Its Roles in Eye Diseases. Front. Nutr. 2022, 9, 1-12, doi:10.3389/fnut.2022.844757
Zhang, S.; Chang, W.; Wu, H.; Wang, Y. H.; Gong, Y. W.; Zhao, Y. L.; Liu, S. H.; Wang, H. Z.; Svatek, R. S.; Rodriguez, R.; et al. Pan-cancer analysis of iron metabolic landscape across the Cancer Genome Atlas. J. Cell. Physiol. 2020, 235, doi:10.1002/jcp.29017

The patent and non-patent references outlined herein are incorporated herein by reference in their entirety. To the extent that any incorporated subject matter contradicts with any disclosure herein, the disclosure herein shall take precedence over the incorporated content.

The present subject matter will be further illustrated in the following examples.

### EXAMPLES

Example 1. Synthesis of a soluble co-polymer chelating composition comprising an active pyridinone metal binding group in the form of MAHMP co-polymerized with vinyl-pyrrolidone using RAFT procedures (U.S. Pat. No. 11,059,785).

### A. Synthesis Procedure

This example composition (Batch no. IS09865-025) shows generally optimized synthesis conditions in terms of obtaining high yield conversion of monomers to co-polymer product and in reference to the following synthesis scheme.

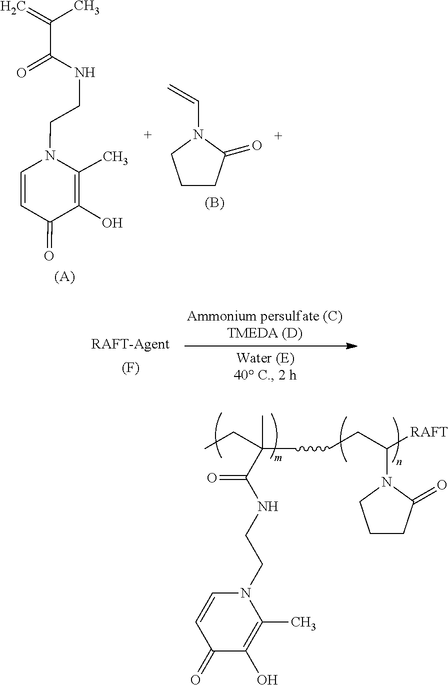

Into a two-neck 50 mL round bottom flask, equipped with a magnetic stirrer and a reflux condenser (3.5 g, 0.0315 mol) N-vinyl-2-pyrrolidone (B) and (0.25 g, 0.00106 mol) MAHMP (A) were dissolved in 4 mL of deionized water (E) and the mixture was stirred for 20 min under N₂. To this mixture (0.136 g, 0.000652 mol) RAFT Agent, 2-ethoxythiocarbonylsulfanyl-2-methyl-propionic acid (ETSPA) (F) and (0.227 g, 0.0.00195 mol) TMEDA (accelerator (D)) were added and the total mixture was degassed by purging N₂ for 20 min. Post degasification, tertiary butyl hydroperoxide (0.35 g, 0.00388 mol) (initiator (C)) was added to the mixture. Thereafter, the mixture was heated to 40° C. and the reaction was continued at the same temperature for 18 h under N₂. After 18 h, the reaction mass was placed on a rotary evaporator to remove water under reduced pressure. The crude mass was dissolved in methanol and filtered to remove any insoluble impurities and the filtrate was concentrated to approximately a 50 weight % polymer solution. This solution was then slowly precipitated with 50 volumes excess MTBE under constant stirring. Thereafter the precipitate was filtered and dried on a rotary evaporator at 50° C. under 35-50 mmHg for 4-6 h, until constant weight of product co-polymer (batch IS09865-025) was observed. The mass yield of the MAHMP-pyrrolidone co-polymer was 80% with respect to the mass of the monomers (A+B) initially supplied to the polymerization reaction and the co-polymer was found to contain a 5.7% MAHMP content as determined by ¹H NMR spectroscopy. The metal chelating co-polymer was found to have a molecular weight (Mw) of approximately 7.3 kDa with a PDI (Mw/Mn) of 1.7 using the GPC analysis methods.

### B: Optimization of MAHMP:NVP Monomer Ratio

In reference to the co-polymer synthesis scheme and example 1A above, a series of test polymerizations were carried out using the procedure of Example A (above) except that the ratio of metal chelating monomer MAHMP (A) to the co-monomer NVP (B) was varied while retaining the overall total monomer (A+B) content at 50 equivalents to the polymerization reaction and while maintaining the other chemical components (C), (D), (E) and (F) the same for each trial. The results in Table 6 below showed that as the amount of MAHMP (A) supplied for polymerization was increased relative to NVP (B) the overall co-polymer yield decreased but the proportion of MAHMP content in the product co-polymer increased. Ideally a co-polymer MAHMP content of 10-20% might be preferred for a chelating co-polymer composition in relation to it having high metal binding capacity, however, retaining high co-polymer yield is also very important for efficiency of monomer use. RAFT mediated polymerization using RAFT agent 4, 2-ethoxythiocarbonylsulfanyl-2-methyl-propionic acid (ETSPA) was found to provide a fairly narrow molecular weight distribution of the co-polymer product with the average Mw of the compositions being somewhat similar at the different MAHMP: NVP conditions. Based on these results it appeared that a MAHMP: NVP ratio of between 1:10 and 1:15 provided the best overall MAHMP content (10-15%) while retaining product yields of approximately 80% and with a Polydispersion Index (PDI) of around 1.4-2.0. Compositions prepared by this synthesis procedure have been designated as DIBI for ease of reference with the other examples provided.

TABLE 6
Optimization of incorporation of
MAHMP into co-polymer composition

| | | | | | Resultant MAHMP- | |
|---|---|---|---|---|---|---|
| | Reactants equivalents | | | | Pyrrolidone Co-polymer | |
| | (See co-polymer | | | | MAHMP | Yield, |
| | synthesis | | | | Mol % | Relative |
| | scheme above) | | | | Incorp | Molecular |
| | A:B | | | | (¹H- | Weight |
| Batch no | [A + B] | F | C | D | NMR) | and PDI |
| IS09865- | 1:20 | 1 | 6 | 3 | 4.6 | Yield: 88%, |
| 042 | [50] | | | | | Mw: 3656 |
| | | | | | | PDI: 1.74 |
| IS09865- | 1:15 | 1 | 6 | 3 | 9.5 | Yield: 80%, |
| 093 | [50] | | | | | Mw: 4799, |
| | | | | | | PDI: 1.9 |
| IS09865- | 1:10 | 1 | 6 | 3 | 15.2 | Yield: 78%, |
| 044 | [50] | | | | | Mw: 3620, |
| | | | | | | PDI: 1.60 |
| IS09865- | 1:5 | 1 | 6 | 3 | 25.6 | Yield: 55%, |
| 041 | [50] | | | | | Mw: 2298, |
| | | | | | | PDI: 1.36 |

From these test syntheses, sample batch IS09865-044 provided a relatively high co-polymer yield (around 80%) while allowing around 15 mol % incorporation of the MAHMP into the co-polymer composition and provided a relative (i.e., relative to linear polystyrene calibration standards using gel exclusion chromatography) product Mw of 3600 Da. Separate true Mw determination of this composition (referred to as DIBI) by laser techniques indicated an actual true Mw of around 9 kDa.

Example 2. Demonstration of the iron binding capacity and iron formation constants of the composition referenced as DIBI in example 1B (Ang et al, 2018 and Gumbau-Brisa et al, 2020).

A. Spectrophotometric characterization of Fe chelation by DIBI

Dry DIBI composition as prepared in Example 1B (30.3 mg, 3.4 mol) was dissolved in 25 mL of MOPS buffer. Aliquots of 1 mL of this solution were placed in vials and increasing volumes of an 8.6 mM Fe(NO₃)₃·9H₂O solution in water were added to a series of vials. A corresponding amount of MOPS buffer was then added to each vial to reach a final reaction volume of 1.1 mL for each vial.

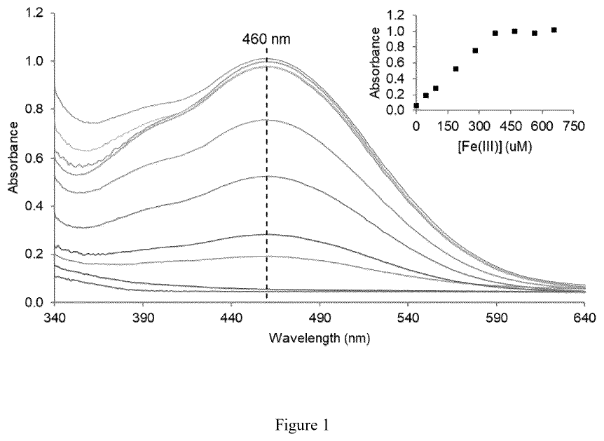

The solutions were shaken gently in a shaker plate for 24 h, at which point the absorbance of 300 μL of each vial was recorded between 350 and 800 nm. The results are shown in FIG. 1.

Plotting the absorption maxima at 460 nm against iron(III) concentration gave a plateau at 371 μM iron(III). This indicated that the 9 MAHMP residues as present on average on each molecule of DIBI, i.e., as measured separately by H¹ nuclear magnetic residence spectroscopy (Ang et al, 2018) were all available for the tris-chelation of iron (i.e., hexadentate binding of 3 iron atoms per molecule of DIBI with the 9 bidentate MAHMP residues, 3 MAHMP moieties for each bound iron atom), resulting in a total iron-binding capacity of 338 mol of iron per gram of DIBI. No precipitate was visible during the iron titration experiments and the coloured films obtained upon solution evaporation, re-dissolved readily in water following weeks of being dry. These results indicate that both DIBI and iron-laden DIBI are soluble in aqueous media.

### B. Iron(III) Binding Affinity of DIBI and MAHMP

To determine the binding affinity for iron(III) of DIBI and its component chelating ligand MAHMP, acidic solutions of the corresponding 1:3 Fe:HPO complex in solutions of constant ionic strength (l=0.1 M) were titrated with NaOH. The titrations were carried out under a wet nitrogen atmosphere to avoid concentration changes due to evaporation. Aliquots of each complex solution were taken periodically during the titration and the UV-vis spectra of the aliquots were collected as a batch once the titration was completed. The pH range studied spanned the equilibrium between bis and tris coordination for the ligands as these are the only two species expected at physiological pH. For Fe₃-DIBI the spectra were collected 24 h after collecting the batch of aliquots. The spectra were processed using HypSpec2014 to determine the iron(III) binding affinity of MAHMP, DIBI and deferiprone (DFP) as reference as summarized in Table 7.

TABLE 7
Iron binding formation constants for deferiprone
(DFP), MAHMP and DIBI at 25° C.

| Chelator | Log β₁ | Log β₂ | Log β₃ |
|---|---|---|---|
| DFP | 15.01(1) | 27.5(7) | 36.7(1) |
| MAHMP | 15.01(1) | 27.9(1) | 38.22(6) |
| DIBI | 15.01(1) | 29.4(3) | 41.05(2) |

*Values in parenthesis are the standard deviations of the last digit

MAHMP appeared similar to DFP as might be expected given their close structural similarities and because both are bidentate chelators, i.e., 3 chelator molecules are required for tris-bidentate coordination of one Fe(III). On the other hand, DIBI has multiple MAHMP chelating monomers (average of 9 per molecule) dispersed along its non-iron-binding polymer backbone. Therefore, formation of an intramolecular tris complex can result from chelating MAHMP monomers in close spatial proximity along the polymer backbone chain or from structural loops bringing the MAHMP residues sufficiently close together. The enhanced iron binding of DIBI is reflected in its higher Log β₂ and especially with its higher Log β₃ formation constants with DIBI displaying Fe binding affinities up to 1000× higher than for either MAHMP or DFP.

Example 3: Resistance of Example 1B, composition (DIBI) bound iron to chemical reduction by dithionite in comparison to reducibility of deferiprone bound iron (Gumbau-Brisa et al, 2020).

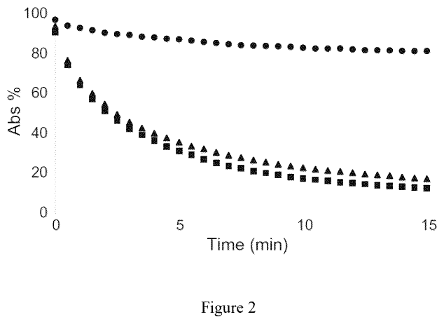

Chemical reduction of iron from Fe(III) to Fe(II) is part of the chemical reductive cycling of iron that drives ROS production in and around cells and also facilities removal of iron from intracellular stores for use elsewhere in the body. Generally iron in extracellular compartments of the body is in the Fe(III) form this helping to limit iron related toxic reactions as free Fe(II) would quickly oxidize to Fe(III) and this could drive, for example, ROS production. Thus, the relative ability of an iron chelator to hold Fe(III) and resist its reduction to Fe(II) would have benefits. Correspondingly, the relative chemical reducibility of Fe(III) to Fe(II) as bound by deferiprone, MAHMP and DIBI was tested. The different iron loaded chelators were first prepared so that iron only sufficient to ensure initial presence of fully coordinated iron(III) was used, i.e. a ratio of three chelator molecules of either deferiprone or MAHMP to each iron(III) atom present. DIBI with its measured MAHMP content was also similarly loaded with iron added just sufficient to satisfy its MAHMP iron binding sites on a similar basis. The iron-chelates were then reacted with dithionite (DT, [S₂O₄]²⁻) a chemical reductant for iron and the relative changes in absorbance intensity at 460 nm was used to monitor for iron reduction with results as shown in FIG. 2.

Dithionite (DT) readily reduced DFP bound iron(III) as shown by the decrease of the intensity of the band at 460 nm for the Fe(DFP)₃ complex solution (FIG. 2). The same reaction was observed for the tris iron(III) complex of MAHMP and spectrophotometric characterization showed similar oxidation rates between Fe(DFP)₃ and Fe(MAHMP)₃. However, under the same conditions, the Fe₃DIBI complex was much more resilient to reduction of its bound iron by DT with only a small loss of intensity of the 460 nm band observed. It is important to note that the spectral change was a decrease in intensity of the 460 nm band, indicative of iron(II) release from the complexes, rather than a wavelength shift of the band maximum, which would indicate iron dissociation by pH change. Accordingly, solutions showed a decrease in absorbance rather than a colour shift from red (colour of the tris complex) to a different colour. Given the similarities between the (HPO) iron binding sites for the three chelators tested, any difference in the behaviour of iron-laden DIBI can be attributed to its tertiary structure and how it holds its bound iron. These results show that DIBI binds iron more tightly and in a more stable fashion with bound iron not readily reducible to Fe(II). On the basis of these results, iron bound by DIBI under physiological conditions as in and around animal cells would be highly stable and thus less available to promote ROS formation from Fe(II)/Fe(II) reduction cycling activity of its bound iron.

Example 4. Demonstration of lowering of intracellular macrophage iron concentrations with DIBI, the composition of example 1B (Ghassemi-Rad, et al. 2022).

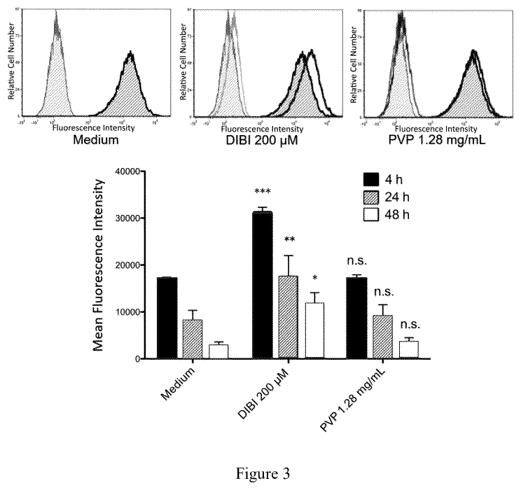

The impact of DIBI on the availability for and intracellular concentration of iron in macrophages was determined by comparing the intracellular pool of labile, i.e. chemically reactive, iron in RAW 264.7 macrophages that were cultured in the absence or presence of 200 μM DIBI. Separate preliminary testing with staining with 7-AAD, a cell viability dye, showed that 200 μM DIBI was not cytotoxic for the RAW 264.7 mouse macrophages or for freshly obtained mouse bone marrow derived macrophages (BMDMs). RAW 264.7 macrophages were maintained at 37° C. in a humidified 5% CO₂ incubator using RPMI 1640 medium supplemented with 5% heat inactivated FBS, 2 mM L-glutamine, 100 μg/mL streptomycin, 100 U/mL penicillin, and 5 mM HEPES buffer (pH 7.4), referred to as complete medium. Cells were passaged using a 25 cm cell scraper. Macrophages were then seeded into a 6-well plate at 2.5×10⁵ cells/well and cultured overnight to allow cells to adhere. The cells were then treated with 0.5 μM calcein-AM and cultured for 30 minutes at 37° C. Cells were then washed with room temperature PBS and treated with complete medium alone or complete medium containing 200 μM DIBI or 1.28 mg/mL polyvinylpyrrolidone (PVP0, i.e. as a control for the polymer backbone of DIBI. Cells that were not exposed to calcein-AM served as an additional control. After 4, 24, and 48 h of culture, cells were harvested and analyzed (1×10⁴ cell counts/sample) using the FL1 channel of a BD FACSCanto™ flow cytometer. The fluorescence of calcein-AM which enters the cells is quenched by intracellular free labile reactive iron so that lower iron levels correspond to higher measured fluorescence. As can be seen in FIG. 3, RAW 264.7 macrophages that were cultured in the presence of DIBI showed a significant decrease in their intracellular pool of labile iron because of iron chelation by DIBI in the culture medium, as determined by flow cytometric analysis of calcein-AM-labeled cells. This effect was specific to the iron-chelating activity of DIBI since an equivalent concentration of PVP of similar molecular weight, i.e., similar to the structural backbone component of the DIBI co-polymer, did not reduce the intracellular pool of labile iron in RAW 264.7 macrophages.

This example shows the polymers disclosed herein may effectively lower intracellular iron levels by binding iron in the immediate extracellular environment of the cell being treated.

Example 5. Reduction of intracellular ROS and extracellular NO production in macrophages with DIBI, the composition of example 1B (Ghassemi-Rad, et al. 2022).

RAW 264.7 cells were seeded into 6-well plates at 2.5×10⁵ cells/well and cultured overnight. The following day, cells were cultured with complete medium alone or complete medium containing 200 μM DIBI or 1.28 mg/mL PVP in the absence or presence of 1 μg/mL bacterial lipopolysaccharide (LPS). Under these conditions macrophage ROS production was induced by addition of LPS a potent inflammatory agent. After 24 h culture, the cells were washed with PBS and resuspended in FBS- and phenol red-free medium containing 10 μM CM-H2DCFDA and incubated in the dark at 37° C. for 30 min to detect intracellular ROS. Cells were then harvested with TrypLE™ Express and washed with room temperature PBS. Cells (1×104 cell counts/sample) were analyzed via the FL1 channel of a BD FACSCalibur™ flow cytometer (BD Biosciences, Mississauga, ON). Data were processed using FCS Express software (version 3.0, De Novo Software, Thornhill, ON).

For separate experiments to measure NO released by macrophages, 100 ng/ml LPS was used to stimulate production and at the end of culture, NO concentrations in the cell-free culture supernatants were measured using the colorimetric Griess assay.

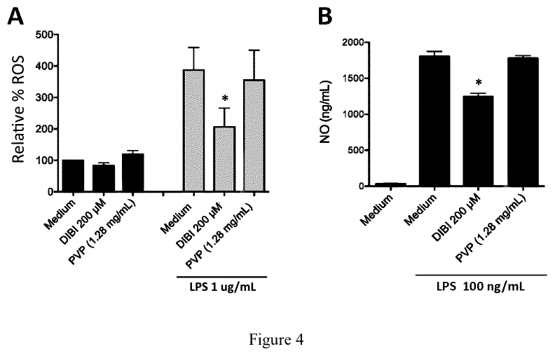

As shown in FIG. 4, LPS-stimulated RAW 264.7 macrophages showed a 4-fold increase in ROS compared to unstimulated control cells. In this experiment 1 μg/mL of LPS was used to trigger vigorous generation of ROS. Treatment with 200 μM DIBI, but not a similar concentration of PVP, reduced ROS production by LPS-stimulated RAW 264.7 macrophages.

ROS that are important for host defence and contribute to inflammatory disease are produced via the electron transport chain of mitochondria, cytochrome P450, and NADPH oxidases. Since the iron-dependent Fenton reaction is important for ROS generation, including generation of the highly toxic hydroxyl radical, it follows that intracellular ROS was reduced in LPS-stimulated macrophages that were treated with DIBI. NO are also generated normally by cells but excesses can contribute to damage to cells. Importantly, DIBI did not completely inhibit ROS or NO production but rather it dampened both excess ROS and NO production. This has significance given macrophage ROS-associated and NO-associated killing of phagocytosed bacterial pathogens is also important in host defence. This example in conjunction with the results of Example 4, shows that the polymers of the present disclosure reduce excess intracellular ROS production and overall NO production by lowering iron levels and therefore lowering excess production as induced by the strong inflammatory LPS agent.

Example 6. Demonstration of reduction of excess inflammatory cytokine production by macrophages with DIBI, the polymer of example 1B (Ghassemi-Rad, et al. 2022).

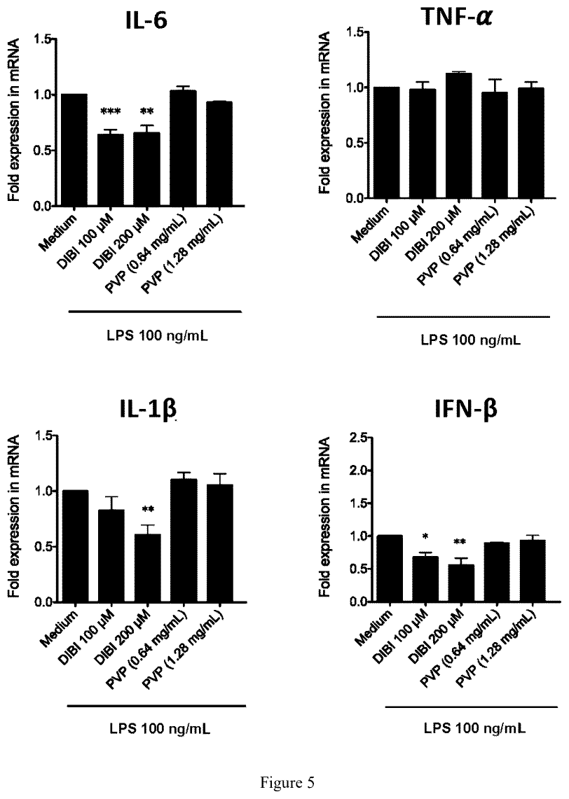

Macrophages prepared as for Example 6 were tested for cellular macrophage mRNA expression for the key inflammatory cytokines IL-1β, IL-6, IFN-β and TNF-α after being cultured for 6 h with medium alone or medium containing the indicated concentrations of DIBI or PVP plus 100 ng/mL LPS to stimulate cytokine expression with results as shown in FIG. 5.

Decreased ROS production by LPS-stimulated macrophages in the presence of DIBI as shown in Example 6, may contribute to the reduction in IL-1β, IL-6, and IFN-β synthesis since mitochondrial ROS promote LPS-induced proinflammatory cytokine synthesis. These various results are consistent with DIBI dampening the excess LPS-induced inflammatory response, i.e., excess ROS and cytokine production, but without fully blocking these. These results also show the PVP carrier material of the DIBI polymer had no effect.

Example 7. Iron dependency of reduction of excess inflammatory cytokine production by macrophages with the polymer DIBI of example 1 (Ghassemi-Rad, et al. 2022).

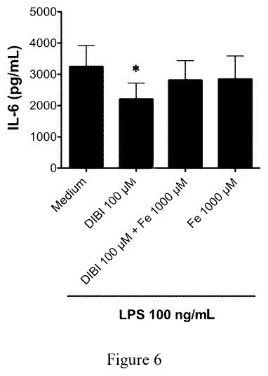

Macrophages were cultured as for Example 6, for 24 h with medium alone or medium containing 200 μM DIBI and/or iron (Fe) as 1000 μM ferric citrate in the absence or presence of 100 ng/mL LPS. Cell-free supernatants were collected, and IL-6 was measured by ELISA with results as shown in FIG. 6.

These results show that the dampening of excess IL-6 cytokine production by DIBI, was directly related to its iron chelating activity in the immediate environment of the macrophages, as addition of excess iron to fully satisfy the added DIBI iron binding capacity reversed the dampening effect of DIBI.

Example 8. Removal of deferiprone bound iron and binding of iron from deferiprone to the DIBI (polymer of example 1B) (Gumbau-Brisa et al, 2020).

DIBI was added to solutions of Fe(DFP)₃ so that both chelators were equimolar with respect to their hydroxypyridinone moiety contents and the resulting solution was stirred at room temperature for 24 h. Dialysis of the reaction solution to separate DIBI from deferiprone on the basis of their different molecular weights. This was achieved by using a dialysis tubing bag with a pore cut-off of 3.5 kDa MW which therefore allowed deferiprone to pass through the membrane but prevented passage of DIBI. After a further 24 h incubation, a coloured dialysis bag retentate solution (DIBI solution) and colourless dialysate DFP solution was obtained. Comparison of the UV-vis spectra of the starting Fe(DFP)₃-DIBI solution and the DIBI dialysis retentate showed that the HPO content (band at 280 nm) had decreased approximately by half, and the iron complex content (broad band at 460 nm) had remained constant. The complex Fe(DFP)₃ was readily dialyzable while Fe₂-3DIBI was retained by the dialysis membrane. This result indicates that DIBI is capable of displacing iron(III) from the coordination environment of DFP and bind the iron displaced from DFP due to DIBI's higher iron binding strength.

Example 9: Removal of iron bound to transferrin and its binding to DIBI, the polymer of Example 1B (as in Gumbau-Brisa et al, 2020).

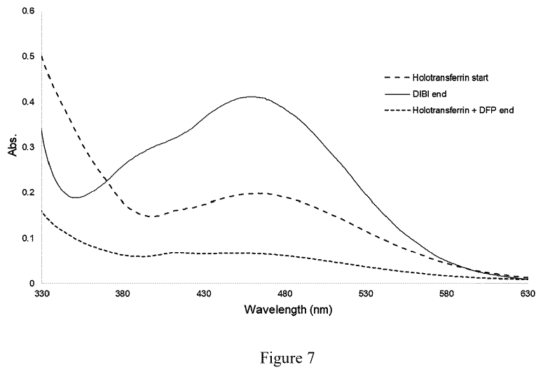

Transferrin has biological relevance as the principle extracellular vertebrate host iron binding, shuttle and iron chaperone protein that would be in play during infection, inflammation and those diseases stemming from iron dysregulation as disclosed in the incident application. A solution of DIBI inside a dialysis tubing bag was dialyzed against a solution of holotransferrin (iron loaded form of transferrin) and DFP in MOPS/bicarbonate buffer at pH 7.4. The dialysis membrane had an exclusion limit 3.5 kDa and ensured the transferrin (MW 80 kDa) and DIBI (MW 9 kDa) remained separated on opposing sides of the membrane while DFP was free to migrate back and forth through the membrane owing to it being a small molecule (MW 139 Da). The mixture was allowed to react at 8° C. for 48 h. After that time the DIBI solution within in the dialysis tubing bag was orange, and the holotransferrin-DFP solution outside of the dialysis bag was pale orange. The DIBI solution was then dialyzed in MOPS/bicarbonate buffer at pH 7.4 for an additional 24 h, after which the UV-vis spectrum of the solution was recorded as shown in FIG. 7. The band at 460 nm in the initial UV-vis spectrum of the holotransferrin solution indicated that transferrin was binding to iron(III) ions. At the end of the experiment the intensity of this band had decreased, indicating a loss of iron(III) from transferrin. After the second dialysis the DIBI solution showed a band at 460 nm, as expected for coordination of iron(III) ions to DIBI.

This example also shows that DIBI has a higher affinity for iron than transferrin. On this basis, DIBI can be expected to augment the natural iron binding and iron chaperoning by transferrin as needed to prevent its inappropriate generation of ROS, i.e., when the iron is present at higher than normal levels as in the diseases disclosed in the incident application.

This example also shows the utility of using a low molecular weight chelator such as deferiprone in conjunction with the disclosed polymers to serve as an iron shuttle to more quickly load the polymer with iron. DIBI removing iron from deferiprone would reduce the possible formation of reactive iron-deferiprone species that could participate in ROS formation due to the incompletely coordinated and therefore reactive iron available on such iron-deferiprone species.

Example 10. Freedom of toxicity of the DIBI polymer of example 1B.

The systemic toxicity for DIBI, the polymer as prepared in example 1B was assessed as administered to both male and female rats by the intravenous route, daily, for a period of 14 consecutive days.

The dose volume administered was 5 mL/kg body weight. Each toxicity test group consisted of 5 rats/sex/group. There were no pre-terminal deaths and no treatment-related clinical signs noted in either male or female rats treated with DIBI at 50, 100 and 200 mg/kg/day. No treatment related effects were noted in mean body weight of either male or female rats receiving DIBI at 50, 100 and 200 mg/kg/day. No changes were observed in food consumption in animals treated with DIBI at 50, 100 and 200 mg/kg/day.

Assessment of clinical chemistry parameters revealed no significant changes in treated rats as compared to non-treated control rats. There were no changes with respect to control rats for hematological parameters including total red blood cell count, total white blood cell count, hematocrit, and hemoglobin.

There were no gross pathological changes or histopathological changes observed in either male or female rats treated with DIBI at 50, 100 and 200 mg/kg/day DIBI based on examination of: the adrenal glands; brain (cerebrum, cerebellum, pons); cecum; colon; duodenum; femur; heart; ileum; jejunum; kidneys; liver; lungs; mesenteric lymph node; rectum; spleen; stomach; sternum; thymus, and testes/ovaries.

Based on the results and with no life threatening pathological observations, the No-Observed-Adverse-Effect-Level (NOAEL) for DIBI was considered to be more than 200 mg/kg/day, i.e. the highest dose tested in this study.

Example 11. Polymers of example 1B (DIBI) supresses excess cytokine production in airway epithelial cells from subjects with Cystic Fibrosis (CF) (Aali et al, 2020).

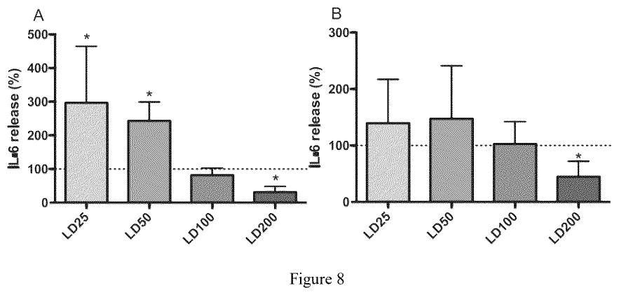

A CF nasal epithelial cell line (JME/CF15), homozygous for the ΔF508 mutation in the CFTR gene were grown in Dulbecco's Modified Eagle's Medium supplemented with 10% fetal bovine serum, 0.088% transferrin, 0.088% T3 hormone (triiodothyronine), 0.0088% EGF (epithelial growth factor); 1.76% hydrocortisone; 0.044% insulin, 0.088% epinephrine and 0.176% adenine. The cells were grown until they reached confluence in T25 tissue culture flasks at 37° C. in a 5% CO₂ atmosphere. Upon confluency, the cell cultures were detached with 0.1% trypsin-EDTA and were seeded into transwells (24 mm diameter with 0.4 μm diameter pores) with both apical and basolateral compartments filled with 2 mL of the described culture media. After one-week and the formation of tight cell monolayers, the apical medium was removed. The monolayers were then maintained under air-liquid interface (ALI) conditions to achieve polarization and differentiation. Following the removal of the apical medium, the medium in the basolateral compartment was changed every two days until the cells achieved of a dry apical surface and were deemed ready for stimulation. Following cell polarization and tight junction (TJ) formation, transwells were designated as either control (medium) or for stimulation with bacterial toxin lipopolysaccharide (LPS) with or without co-treatment with DIBI (LPS+DIBI) or DIBI alone (DIBI). Stimulation with LPS (from Escherichia coli, serotype 026:B6; Sigma-Aldrich, Oakville, ON) was performed in fresh phenol red-free medium at concentrations of 100, 200, or 300 ng/mL for bidirectional stimulations of the cells. Once optimal LPS stimulation conditions were established (dose for 24 hours), 200 ng/mL LPS exposure was found to be optimal. Experimental groups were tested: control (medium only); LPS (200 ng/mL); LPS+DIBI (25, 50, 100 or 200 μM) and, DIBI only (25, 50, 100 or 200 μM). The appropriate stimulation medium was applied to both the apical and basolateral surfaces at volumes of 0.5 mL and 2.5 mL, respectively. The cells were left to incubate for 24 hours in a 37° C. and 5% CO₂ environment before cell harvesting. Enzyme-linked immunosorbent assay (ELISA) kits were used to measure the IL-6 levels in the apical and basolateral cell culture supernatants. Monoclonal human IL-6 antibodies (Invitrogen, Carlsbad, CA) were used. The cytokine levels were normalized to the total protein concentration of the lysed cells, as measured via the Bradford Colorimetric Assay. FIG. 8 shows the stimulation of IL-6 cytokine production and release caused by LPS and its suppression of excess IL-6 release by DIBI from both the apical (A) and basolateral (B) cell surfaces.

IL-6 is a key cytokine associated with pro-inflammatory responses and along with this there is increased ROS production as shown for macrophages in examples 5 and 6. It is known for CF airway cells that ROS damage as mediated by excess, i.e., above normal amounts, of reactive iron is part of the pathogenesis of CF disease, i.e., as related to ROS mediated cell and tissue damage in the airways of CF subjects. This example shows that DIBI which takes up excess, above normal amounts of reactive iron, dampness the excess inflammatory response provoked by LPS in a dose dependent manner, as related to cytokine production. Associated with this there would be a similar dampening in ROS activity as demonstrated for the iron specific dampening by DIBI of excess IL-6 and ROS activity in macrophages as shown in FIGS. 4, 5 and 6. LPS is a potent inflammatory agent released from the cell walls of Gram negative bacteria such as Pseudomonas aeruginosa which has been shown to typically colonize the airways of CF subjects.

What has been described is merely illustrative of the application of the principles of the disclosure. However, it will be apparent to a person skilled in the art that a number of variations and modifications can be made without departing from the scope of the following claims.

1. A method for treatment of a subject having a disease with an iron-mediated pathology, comprising:
administering an iron-chelating polymer comprising a reaction product of:
a first monomer unit and a second monomer units polymerized by a reversible addition-fragmentation chain transfer mechanism with the use of a suitable addition-fragmentation chain transfer agent;
wherein the first monomer unit is represented by Compound (I): 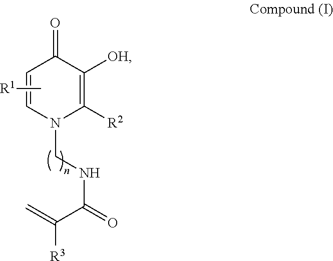
wherein:
R¹ is independently selected from the group consisting of H, alkyl optionally substituted with one of more of O, N or S;
R² is independently selected from the group consisting of H, alkyl optionally substituted with one of more of O, N or S;
R³ is independently selected from the group consisting of H, alkyl optionally substituted with one of more of O, N or S; and
n is 1 to 12;
wherein the second monomer unit is independently selected from the group consisting of 1-vinyl-2-pyrrolidone, acrylic acid, methyl methacrylate, N,N-dimethyl-acrylamide, ethyl methacrylate, N-vinyl imidazole and styrene,
wherein the iron-chelating polymer is dissolved in an aqueous medium,
wherein the iron-chelating polymer has a molecular weight, prior to chelation, of at least about 1500 Da, and
wherein the iron-chelating polymer comprises one or more intramolecular hexadentate ligands for chelating iron, and
optionally further comprising an iron chelating compound having a molecular weight of less than about 1500 Da optionally selected from deferoxamine, deferasirox, deferiprone, SP-420, FBS701, and MAHMP,
wherein the disease is disease is an autoimmune, metabolic, inflammatory or neurological disease optionally selected from one or more of: Systemic Lupus Erythematosus Associated Nephritis, Rheumatoid Arthritis, Parkinson's Disease, Alzheimer's Disease, Fredreich's Ataxia, Amyotrophic Lateral Sclerosis, Fanconi's and related kidney disease, Type 2 Diabetes Mellitus, Hemochromatosis, Thalassemia, Macular Degeneration Eye Disease, and Cardiovascular Disease.

2. (canceled)

3. The method of claim 1, wherein the one or more suitable metal binding chemical groups are a hydroxypyridinone: 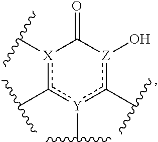
wherein X, Y and Z are independently N or C such that:
when X is N, Y and Z are C,
when Y is N, X and Z are C, and
when Z is N, X and Y are C.

4. The method of claim 1, wherein:
R¹ is H;
R² is methyl;
R³ is methyl; and
n is 1 to 6, optionally n is 2.

5. The method of claim 1, wherein the first monomer unit is represented by Compound (II):

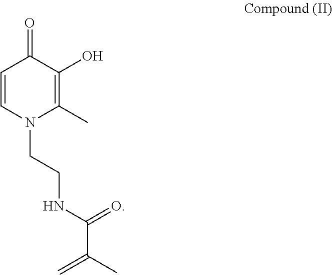

6. The method of claim 1, wherein the second monomer unit is 1-vinyl-2-pyrrolidone.

7. The method of claim 1, wherein the second monomer unit is N,N-dimethyl-acrylamide.

8. The method of claim 1, wherein the suitable addition-fragmentation chain transfer agent is independently selected from the group consisting of 2-ethoxythiocarbonylsulfanyl-propionic acid ethyl ester and 2-ethoxythiocarbonylsulfanyl-2-methyl-propionic acid.

9. The method of claim 1, wherein a residue of the addition-fragmentation chain transfer agent is removed in whole or in part from the iron-chelating polymer after polymerization.

10. The method of claim 1 wherein the iron-chelating polymer lowers an extracellular concentration of free iron and optionally an intracellular concentration of free iron.

11-15. (canceled)

16. An iron-chelating polymer for use in the treatment of a subject having a disease with an iron-mediated pathology, wherein:
the iron-chelating polymer comprises a reaction product of:
a first monomer unit and a second monomer units polymerized by a reversible addition-fragmentation chain transfer mechanism with the use of a suitable addition-fragmentation chain transfer agent;
wherein the first monomer unit is represented by Compound (I): 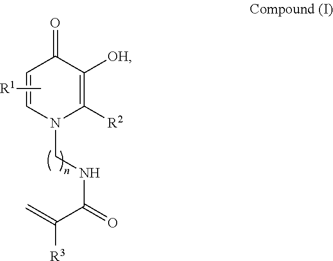
wherein:
R¹ is independently selected from the group consisting of H, alkyl and alkyl optionally substituted with one of more of O, N or S;
R² is independently selected from the group consisting of H, alkyl and alkyl optionally substituted with one of more of O, N or S;
R³ is independently selected from the group consisting of H, alkyl and alkyl optionally substituted with one of more of O, N or S; and
n is 1 to 12;
wherein the second monomer unit is independently selected from the group consisting of 1-vinyl-2-pyrrolidone, acrylic acid, methyl methacrylate, N,N-dimethyl-acrylamide, ethyl methacrylate, N-vinyl imidazole and styrene,
wherein the iron-chelating polymer is dissolved in an aqueous medium,
wherein the iron-chelating polymer has a molecular weight, prior to chelation, of at least about 1500 Da, and
wherein the iron-chelating polymer comprises one or more intramolecular hexadentate ligands for chelating iron, and
optionally further comprising an iron chelating compound having a molecular weight of less than about 1500 Da optionally selected from deferoxamine, deferasirox, deferiprone, SP-420, FBS701, and MAHMP,
wherein the disease is disease is an autoimmune, metabolic, inflammatory or neurological disease optionally selected from one or more of: Systemic Lupus Erythematosus Associated Nephritis, Rheumatoid Arthritis, Parkinson's Disease, Alzheimer's Disease, Fredreich's Ataxia, Amyotrophic Lateral Sclerosis, Fanconi's and related kidney disease, Type 2 Diabetes Mellitus, Hemochromatosis, Thalassemia, Macular Degeneration Eye Disease, and Cardiovascular Disease.

17. The iron-chelating polymer of claim 16, wherein the one or more suitable metal binding chemical groups are a hydroxypyridinone: 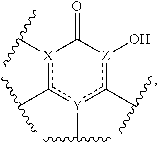
wherein X, Y and Z are independently N or C such that:
when X is N, Y and Z are C,
when Y is N, X and Z are C, and
when Z is N, X and Y are C.

18. The iron-chelating polymer of claim 16, wherein:
R¹ is H;
R² is methyl;
R³ is methyl; and
n is 1 to 6.

19. The iron-chelating polymer of claim 18, wherein n is 2.

20. The iron-chelating polymer of claim 16, wherein the first monomer unit is represented by Compound (II):

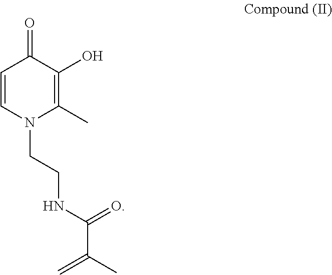

21. The iron-chelating polymer of claim 16, wherein the second monomer unit is 1-vinyl-2-pyrrolidone.

22. The iron-chelating polymer of claim 16, wherein the second monomer unit is N,N-dimethyl-acrylamide.

23. The iron-chelating polymer of claim 16, wherein the suitable addition-fragmentation chain transfer agent is independently selected from the group consisting of 2-ethoxythiocarbonylsulfanyl-propionic acid ethyl ester and 2-ethoxythiocarbonylsulfanyl-2-methyl-propionic acid.

24. The iron-chelating polymer of claim 16, wherein a residue of the addition-fragmentation chain transfer agent is removed in whole or in part from the iron-chelating polymer after polymerization.

25. The iron-chelating polymer of claim 16, wherein the iron-chelating polymer lowers an extracellular concentration of free iron and optionally lowers an intracellular concentration of free iron.

26-31. (canceled)
